(19) United States
(12) Patent Application Publication
(10) Pub. No.: US 2012/0164705 A1
(43) Pub. Date: Jun. 28, 2012

(54) Metabolic Switch

(75) Inventor: Nikos Basil Reppas, Brookline, MA (US)

(73) Assignee: JOULE UNLIMITED TECHNOLOGIES, INC., Cambridge, MA (US)

(21) Appl. No.: 13/304,034

(22) Filed: Nov. 23, 2011

Related U.S. Application Data
(60) Provisional application No. 61/417,105, filed on Nov. 24, 2010.

(51) Int. Cl. C12P 7/06; C12N 1/19; C12N 1/15; C12N 1/13; C12N 5/10; C12N 1/21

(52) U.S. Cl. 435/161; 435/419; 435/252.33; 435/252.3; 435/252.31; 435/254.3; 435/252.35; 435/254.5; 435/253.3; 435/257.2; 435/412; 435/254.21; 435/254.23; 435/254.2

(57) ABSTRACT

The present invention provides compositions and methods for controlling biosynthetic pathways using a metabolic switch in microorganisms. Photoautotrophs are developed to be auxotrophic for certain exogenous compounds such as lipoic acid and/or a fixed nitrogen source. Depletion of the exogenous compound results in the carbon flux to be diverted to preferred metabolic pathways.

### CROSS REFERENCE TO RELATED APPLICATIONS

This application claims priority from U.S. Provisional Application 61/417,105, filed on Nov. 24, 2010, the disclosure of which is incorporated by reference herein for all purposes.

### SEQUENCE LISTING

The instant application contains a Sequence Listing which has been submitted in ASCII format via EFS-Web and is hereby incorporated by reference in its entirety. Said ASCII copy, created on Nov. 23, 2011, is named 19932US_Sequence_Listing.txt and is 15,363 bytes in size.

### FIELD

The disclosure relates to compositions and methods employing microorganisms to produce carbon-based products of interest.

### BACKGROUND

Microorganisms have long been employed to generate desirable products useful for human application and consumption. More recently, microorganisms are being specifically engineered for industry and research to synthesize biomolecules that are otherwise prohibitively expensive to manufacture when utilizing chemical methodologies. Synthesizing biomolecules in microorganisms most commonly incorporates “on or off” expression systems wherein synthesis begins upon addition of a chemical to the growth medium, with cellular carbon and nutrients siphoned away from functioning metabolic pathways. However, this carbon siphoning lowers global cellular production efficiency and productivity because the nutrient and cellular carbon pool remains effectively the same, but is required to be distributed to yet another activated metabolic biosynthesis pathway. New methods are sought continuously by which to make the production of industrially important biomolecules from engineered microorganisms more efficient and less costly. Furthermore, microorganisms offer a cost effective way to produce common industrially important chemicals. Yeast, for example, is used for the fermentation of sugars to ethanol, bacteria such as Escherichia coli are engineered to over-produce biomolecules for industry and research, and photosynthetic cyanobacteria are being used for the generation of alternative fuels, wastewater treatment, food, enzymes and pharmaceuticals.

Photosynthesis is a process by which biological entities utilize sunlight and CO₂ to produce sugars for energy. Photosynthesis, as naturally evolved, is an extremely complex system with numerous and poorly understood feedback loops, control mechanisms, and process inefficiencies. This complicated system presents likely insurmountable obstacles to either one-factor-at-a-time or global optimization approaches (Nedbal et al., Photosynth Res., 93(1-3):223-34 (2007); Salvucci et al., Physiol Plant., 120(2):179-186 (2004); Greene et al., Biochem J., (2007) 404(3):517-24).

Existing photoautotrophic organisms (i.e., plants, algae, and photosynthetic bacteria) are poorly suited for industrial bioprocessing and have therefore not demonstrated commercial viability for this purpose. Such organisms have slow doubling time (3-72 hrs) compared to industrialized heterotrophic organisms such as Escherichia coli (20 minutes), reflective of low total productivities. In addition, techniques for genetic manipulation (knockout, over-expression of transgenes via integration or episomic plasmid propagation) are inefficient, time-consuming, laborious, or non-existent.

### SUMMARY

The invention described herein embodies an isolated host cell comprising at least one control element, auxotrophy for at least one exogenous compound, a heterologous metabolic pathway, and at least one second metabolic pathway, wherein the exogenous compound controls activity of said control element, and the control element controls carbon flux through a metabolic junction shared by the metabolic pathways.

In one embodiment, a heterologous metabolic pathway directs the biosynthesis of carbon-based products of interest.

In one embodiment, a heterologous metabolic pathway directs the biosynthesis of ethanol from pyruvate wherein a host cell produces ethanol upon depletion of an exogenous compound.

In another embodiment, the at least one second metabolic pathway is an engineered metabolic pathway.

In one embodiment, heterologous metabolic pathways for the biosynthesis of fatty acid derivatives from acetyl-coA are controlled by a metabolic switch, wherein said host cell produces fatty derivatives upon depletion of the exogenous compound.

In one embodiment, a heterologous metabolic pathway comprises biosynthesis of an alkane from acetyl-coA.

In a related embodiment, metabolic pathways for biosynthesis of fatty acid derivatives from acetyl-coA and biosynthesis of ethanol from pyruvate are controlled by one or more metabolic switches.

In a related embodiment, a host cell produces ethanol upon depletion of an exogenous compound affecting the metabolic pathway for ethanol biosynthesis and produces fatty acid derivatives upon depletion of an exogenous compound affecting the metabolic pathway for fatty acid derivatives biosynthesis.

In an embodiment of the present invention, a metabolic pathway comprises at least one heterologous gene for the biosynthesis of ethanol from pyruvate, and/or at least one endogenous gene for metabolizing pyruvate to ethanol.

In an embodiment of the present invention, a metabolic switch comprises at least one genetic element selected from the group consisting of a heterologous nitrite reductase promoter Pₙᵢᵣ, an endogenous nitrite reductase promoter Pₙᵢᵣ., and SEQ ID NO: 1.

In a related embodiment, a metabolic switch comprises at least one protein selected from the group consisting of AceF, LplA, Pdh, AceE, pyruvate dehydrogenase, dihydrolipoyl transacetylase and dihydrolipoyl dehydrogenase.

In a similarly related embodiment, a metabolic switch comprises at least one genetic element selected from the group consisting of a heterologous nitrite reductase promoter an endogenous nitrite reductase promoter Pₙᵢᵣ., and SEQ ID NO: 1 and at least one protein selected from the group consisting of AceF, LplA, Pdh, AceE, pyruvate dehydrogenase, dihydrolipoyl transacetylase and dihydrolipoyl dehydrogenase.

The present invention can be in hosts cells selected from eukaryotic plants, industrially important organisms including Xanthomonas spp., Escherichia coli, Corynebacterium spp., Lactobacillus spp., Aspergillus spp., Streptomyces spp., Acetobacter spp., Penicillin spp., Bacillus spp., Pseudomonad spp., Clostridium spp., Zymomonas spp., Salmonella spp., Serratia spp., Erwinia spp., Klebsiella spp., Shigella spp., Enteroccoccus spp., Alcaligenes spp., Paenibacillus spp., Arthrobacter spp., Brevibacterium spp., algae, cyanobacteria, green-sulfur bacteria, green non-sulfur bacteria, purple sulfur bacteria, purple non-sulfur bacteria, extremophiles, yeast, fungi, engineered organisms thereof, and synthetic organisms.

The present invention can be in host cells that are light dependent or fix carbon, and/or releases, permeates or exports carbon-based product of interest from the host cell, including, without limitation, ethanol.

In a preferred embodiment, a metabolic pathway comprises a heterologous alcohol dehydrogenase (Adh) and a heterologous pyruvate decarboxylase (Pdc).

In a related embodiment, a heterologous Adh is selected from any one or more of Zymomonas mobilis adhII, Z. mobilis adhII TS42, Z. mobilis adhB or Moorella sp. HUC22-1 adhA and combinations thereof.

In another related embodiment, a heterologous Pdc is selected from Zymobacter palmae or Zymomonas mobilis and combinations thereof.

In one embodiment, a host cell is attenuated in lactate dehydrogenase, pyruvate formate lyase and/or pyruvate:ferredoxin oxidoreductase activities.

In a related embodiment a host cell is auxotrophic for lipoic acid, and/or the cell comprises a heterologous lipoylation gene product.

In a related embodiment, a heterologous lipoylation is achieved from Escherichia coli gene product LplA and/or from lipoyl (octanoyl) transferase (EC 2.3.1.181) and lipoyl synthase (EC 2.8.1.8).

In another embodiment a host cell is attenuated for acyl-ACP synthetase protein (EC 6.2.1.20), LipB, LipA1 or LipA2 gene products and any combinations thereof.

In yet another embodiment of the present invention, a host cell further comprises heterologous sodium:solute symporter, selected from, but not limited to, mammalian SMVT, Escherichia coli PanF and Escherichia coli YipK.

In another related embodiment, a host cell further comprises a heterologous lipoate transport system of, but not limited to, LipT, EcfA1, EcfA2 and Efc2 or homologues thereof.

In one embodiment, a host cell is auxotrophic for a fixed nitrogen source and comprises a heterologous lipoamidase (Lpa), heterologous alcohol dehydrogenase (Adh) and a heterologous pyruvate decarboxylase (Pdc).

In a related embodiment heterologous Lpa is selected from Enterococcus faecalis, NCBI Accession #AAU94937, and Enterococcus faecalis, NCBI Accession #AAU94937 truncated at any one of amino acid position 450-490.

In a specific related embodiment a heterologous Lpa activity selected from Enterococcus faecalis, NCBI Accession #AAU94937 truncated at amino acid position 471.

In another related embodiment, heterologous Lpa expression is controlled by an endogenous and/or heterologous nitrite reductase (Pₙᵢᵣ) promoter.

In a specific related embodiment, heterologous Lpa expression is controlled by a heterologous nitrite reductase promoter selected from the nitrate assimilation operon of Synechococcus sp. strain PCC 7942 or is controlled by SEQ ID NO: 1.

In a related embodiment, a host cell is attenuated in LipA1, LipA2, Pdh, subunits of Pdh, AceF, AceE, NifJ, LdhA or Pps activities and combinations thereof.

In one embodiment is a method for the production of carbon-based products of interest, comprising (a) culturing a host cell with at least one control element, a heterologous metabolic pathway, and auxotrophy for at least one auxotrophic compound; (b) depleting the exogenous compound from the culture; and (c) controlling carbon flux through the metabolic junction shared by the metabolic pathways. In one related embodiment, the at least one second metabolic pathway is an engineered metabolic pathway. In a related embodiment, the method further comprises the host cell attenuating acetyl-CoA production upon depletion of said exogenous compound of step (b). In another related embodiment, the method further comprises the host cell attenuates acetyl-CoA production and initiates ethanol production concomitant with depletion of said exogenous compound of step (b).

In one embodiment is a method for the biosynthesis of carbon-based products of interest in a host cell, comprising (a) providing an engineered host cell auxotrophic, with at least one control element, at least one heterologus metabolic pathway, at least one second metabolic pathway, and a shared metabolic junction, wherein the exogenous compound controls the activity of the control element, and the control element controls carbon flux through the metabolic junction to a preferred metabolic pathway; (b) culturing the host cell in a growth medium with the exogenous compound; and (c) depleting the exogenous compound from the culture.

In one embodiment of the method, the at least one second metabolic pathway is an engineered pathway.

The present methods of the invention described herein embody host cells selected from eukaryotic plants, industrially important organisms including Xanthomonas spp., Escherichia coli, Corynebacterium spp., Lactobacillus spp., Aspergillus spp., Streptomyces spp., Acetobacter spp., Penicillin spp., Bacillus spp., Pseudomonad spp., Clostridium spp., Zymomonas spp., Salmonella spp., Serratia spp., Erwinia spp., Klebsiella spp., Shigella spp., Enteroccoccus spp., Alcaligenes spp., Paenibacillus spp., Arthrobacter spp., Brevibacterium spp., algae, cyanobacteria, green-sulfur bacteria, green non-sulfur bacteria, purple sulfur bacteria, purple non-sulfur bacteria, extremophiles, yeast, fungi, engineered organisms thereof, and synthetic organisms.

The present methods invention embody host cells that are light dependent or fix carbon, produce-carbon based product of interest including, without limitation, ethanol, and releases, permeates or exports the carbon-based product of interest from the host cell.

In one embodiment of the described methods the metabolic pathway of the host cell comprises a heterologous alcohol dehydrogenase (Adh) and a heterologous pyruvate decarboxylase (Pdc), and includes, but is not limited to, Adh selected from Z. mobilis adhII, Z. mobilis adhII TS42, Z. mobilis adhB or Moorella sp. HUC22-1 adhA and combinations thereof, and heterologous Pdc selected from Z. palmae or Z. mobilis and combinations thereof.

In related embodiments of the described methods, the host cell further comprises attenuated lactate dehydrogenase, pyruvate formate lyase or pyruvate:ferredoxin oxidoreductase and combinations thereof.

In another embodiment of the described methods, the host cell comprises auxotrophy for lipoic acid, a heterologous lipoylation gene product including, without limitation, Escherichia coli LplA, lipoyl (octanoyl) transferase (EC 2.3.1.181) and lipoyl synthase (EC 2.8.1.8).

In a related embodiment of the methods of the present invention the host cell further comprises attenuated acyl-CoA synthetase, attenuated LipB, LipA1 and/or LipA2, and combinations thereof.

In another related embodiment of the methods described herein, the host cell comprises a heterologous sodium:solute symporter, including, without limitation, mammalian SMVT, Escherichia coli PanF and Escherichia coli YipK, and a heterologous lipoate transport system, including, without limitation, LipT, EcfA1, EcfA2 and Efc2.

In one embodiment of the methods described herein, the host cell comprises auxotrophy for a fixed nitrogen source, a heterologous lipoamidase (Lpa), a heterologous alcohol dehydrogenase (Adh) and a heterologous pyruvate decarboxylase (Pdc), wherein the heterologous Lpa is selected from Enterococcus faecalis, NCBI Accession #AAU94937, and Enterococcus faecalis, NCBI Accession #AAU94937 truncated at any one of amino acid position 450-490.

In a related embodiment of the methods described herein, the heterologous Lpa is expression controlled by an endogenous or heterogenous nitrite reductase promoter, the nitrate assimilation operon of Synechococcus sp. strain PCC 7942, or by SEQ ID NO: 1.

In embodiments utilizing nitrate sources, the nitrate compounds comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% of the exogenous fixed nitrogen source, and urea comprises the remaining proportion of exogenous fixed nitrogen source.

In embodiments of the present invention, the host cell has ethanol production rates in a stationary growth phase at least the same as in a linear growth phase of said host cell.

In embodiments of the present invention, a host cell's ethanol production is at least 50%, at least 55%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, least 85%, at least 90%, at least 95%, at least 100% of biomass productivity of a wild type strain from which said host cell is derived.

In embodiments of the present invention, a host cell has ethanol production rates of at least 100 mg/L culture medium/hour, at least 125 mg/L culture medium/hour, at least 150 mg/L culture medium/hour, at least 175 mg/L culture medium/hour, at least 200 mg/L culture medium/hour.

In other embodiments of the present invention is a method wherein a host cell has ethanol production rates in a stationary growth phase at least the same as in a linear growth phase of said host cell.

In other embodiments of the present invention is a method wherein a host cell has ethanol production is at least 50%, at least 55%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, least 85%, at least 90%, at least 95%, at least 100% of biomass productivity of a wild type strain from which said host cell is derived.

In other embodiments of the present invention is a method wherein a host cell has ethanol production rates of at least 100 mg/L culture medium/hour, at least 125 mg/L culture medium/hour, at least 150 mg/L culture medium/hour, at least 175 mg/L culture medium/hour, at least 200 mg/L culture medium/hour.

In certain embodiments is a host cell having the productivity of ethanol at a transition mid-point between a linear growth phase and a stationary growth phase at least as much as the productivity of ethanol during the linear growth phase.

In other embodiments is a method wherein a host cell has productivity of ethanol at a transition mid-point between a linear growth phase and a stationary growth phase at least as much as the productivity of ethanol during the linear growth phase.

In certain embodiments is a host cell having productivity of ethanol at a transition mid-point between a linear growth phase and a stationary growth phase at least 95%, at least 90%, at least 85%, at least 80%, at least 75%, at least 70%, at least 65%, at least 60%, at least 55%, at least 50%, at least 45%, at least 40%, at least 35%, at least 30%, at least 25%, at least 20% the productivity of ethanol during the linear growth phase.

In other embodiments is a method wherein a host cell has productivity of ethanol at a transition mid-point between a linear growth phase and a stationary growth phase is at least 95%, at least 90%, at least 85%, at least 80%, at least 75%, at least 70%, at least 65%, at least 60%, at least 55%, at least 50%, at least 45%, at least 40%, at least 35%, at least 30%, at least 25%, at least 20% the productivity of ethanol during the linear growth phase.

In certain embodiments is a host cell wherein the host cell further comprises a photosynthetic efficiency of at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, at least 1000% more than the photosynthetic efficiency of the host cell without a metabolic switch.

In other embodiments is a method wherein a host cell further comprises a photosynthetic efficiency of at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, at least 1000% more than a photosynthetic efficiency of the host cell without a metabolic switch.

In one embodiment is a host cell comprising a control element and a heterologous metabolic pathway, wherein said host cell is auxotrophic for an exogenous compound, said compound controls activity of said control element, and said control element controls carbon flux through a metabolic junction shared by said heterologous metabolic pathway and at least one second metabolic pathway. In one aspect, the at least one second metabolic pathway of the host cell is an engineered metabolic pathway.

### BRIEF DESCRIPTION OF THE FIGURES

FIG. 1: Schematic showing pyruvate in a central role of a metabolic junction where it serves as a precursor to ethanol production or metabolism to acetyl-CoA, and associated enzymes of alternative metabolic pathways.

FIG. 2: Cellular pathways for exogenous lipoic acid import and activation of protein targets.

FIG. 3: Alternative pathways for an exogenous compound, lipoic acid, entering a host cell across the plasma membrane.

FIG. 4: Schematic showing pyruvate and acetyl-CoA in central roles of a metabolic junction wherein each serves as a precursor to carbon-based products including ethanol, fatty acids, fatty acid esters, alkanes and alkenes, and associated enzymes of metabolic biosynthetic pathways.

FIG. 5: Graph of (A) dry cell weight and (B) ethanol produced over time by JCC138 in the presence or absence of lipoic acid, JCC1518 in the presence or absence of lipoic acid, and JCC1518 lipoic acid auxotroph in the presence of lipoic acid.

### DETAILED DESCRIPTION

### Abbreviations and Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a cell” includes one or a plurality of such cells, and reference to “comprising the thioesterase” includes reference to one or more thioesterase peptides and equivalents thereof known to those of ordinary skill in the art, and so forth. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Accession Numbers: The accession numbers throughout this description are derived from the NCBI database (National Center for Biotechnology Information) maintained by the National Institute of Health, U.S.A. The accession numbers are as provided in the database on Feb. 1, 2008.

Amino acid: Triplets of nucleotides, referred to as codons, in DNA molecules code for amino acid in a peptide. The term codon is also used for the corresponding (and complementary) sequences of three nucleotides in the mRNA into which the DNA sequence is transcribed.

Attenuate: The term as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. Attenuation as applied to a nucleotide sequence encoding a gene or gene expression control sequence also refers to attenuation of the protein, and attenuation of a protein also refers to attenuation of the corresponding gene encoding the protein and/or the gene expression control sequence. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

Autotroph: Autotrophs (or autotrophic organisms) refers to organisms that produce complex organic compounds from simple inorganic molecules and an external source of energy, such as light (a “photoautotroph,” or alternatively referred to, “photoautotrophic host cell”) or chemical reactions of inorganic compounds.

Auxotroph: Auxotrophs (or auxotrophic organisms) refers to organisms that do not have the ability to synthesize one or more particular compounds that are required for growth, and/or metabolic sustainability sufficient for the organism to maintain a living state or otherwise maintain viability, and is otherwise unable to synthesize or provide to itself intra-cellularly because of natural or genetic engineering means.

Biofuel: A biofuel refers to any fuel that is derived from a biological source. Biofuel refers to one or more hydrocarbons, one or more alcohols, one or more fatty esters or a mixture thereof. Preferably, liquid hydrocarbons are used.

Biosynthetic pathway: Also referred to as “metabolic pathway,” a biosynthetic pathway refers to a set of anabolic or catabolic biochemical reactions for converting (transmuting) one chemical species into another. Generally, a metabolic pathway is the set of biochemical reactions encompassing the structural and/or chemical transformations connecting a single substrate to an end-product formation with the necessary enzymatic reactions for its occurrence. For example, a hydrocarbon biosynthetic pathway refers to the set of biochemical reactions that convert substrates and/or metabolites to hydrocarbon product-like intermediates and then to hydrocarbons or hydrocarbon products. Anabolic pathways involve constructing a larger molecule from smaller molecules, a process requiring energy. Catabolic pathways involve breaking down of larger molecules, often releasing energy.

Carbon-based product of interest: A carbon-based product of interest (or carbon-based product) refers to, without limitation or implication that the scope of the claims are limited to the examples set forth herein, desirable end-products or metabolites produced by a biosynthetic pathway of an isolated host cell. The end products or metabolites include, but are not limited to, alkanes (propane, octane), alkenes (ethylene, 1,3-butadiene, propylene, olefins, alkenes, isoprene, lycopene, terpenes) aliphatic and aromatic alkane and alkene mixtures (diesel, jet propellant 8 (JP8)), alkanols and alkenols (ethanol, propanol, isopropanol, butanol, fatty alcohols, 1,3-propanediol, 1,4-butanediol, polyols, sorbitol, isopentenol), alkanoic and alkenoic acids (acrylate, acrylic acid, adipic acid, itaconic acid, itaconate, docosahexaenoic acid, (DHA), omega-3 DHA, malonic acid, succinate, omega fatty acids), hydroxy alkanoic acids (citrate, citric acid, malate, lactate, lactic acid, 3-hydroxypropionate, 3-hydroxypropionic acid (HPA), hydroxybutyrate), keto acid (levulinic acid, pyruvi acid), alkyl alkanoates (fatty acid esters, wax esters, c-caprolactone, gamma butyrolactone, γ-valerolactone), ethers (THF), amino acids (glutamate, lysine, serine, aspartate, aspartic acid, glutamic acid, leucine, isoleucine, valine), lactams (pyrrolidones, caprolactam), organic polymers (terephthalate, polyhydroxyalkanoates (PHA), poly-beta-hydroxybutyrate (PHB), rubber), isoprenoids (lanosterol, isoprenoids, carotenoids, steroids), pharmaceuticals/multi-functional group molecules (ascorbate, ascorbic acid, paclitaxel, docetaxel, statins, erythromycin, polyketides, peptides, 7-aminodeacetoxycephalosporanic acid (7-ADCA)/cephalosporin) and metabolites (acetaldehyde).

Cataplerosis: Cataplerosis or “cataplerotic” refers to a metabolic pathway(s) that use as substrates chemical intermediates and/or species of other metabolic pathways, thereby diverting those substrates or intermediates away from the other metabolic pathways. For example, pyruvate can be considered an intermediate in a metabolic pathway for acetyl-CoA formation. However, pyruvate can also serve as a substrate for lactate dehydrogenase, pyruvate decarboxylase and/or enzymes for the formation of some amino acids. Therefore, as used herein, metabolic pathways that divert a substrate or intermediate away from a metabolic pathway synthesizing a carbon-based product of interest are considered cataplerotic. In contrast, metabolic pathways that divert a substrate or intermediate back into a metabolic pathway synthesizing a carbon-based product of interest is an anaplerotic metabolic pathway.

Control Element: A control element, or metabolic control element, refers to a genetic element or protein capable of being directly or indirectly acted upon by an exogenous compound to attenuate or activate, directly or indirectly, at least one metabolic pathway. The carbon flux through a metabolic pathway acted on by the control element (genetic element or protein) can become redirected to at least one second metabolic pathway. Depletion of an exogenous compound, which can be the process of removal, partial removal or complete removal, of the exogenous compound from the cell, allows the carbon flow to be directed back to the metabolic pathway from which the carbon flux was diverted.

Deletion: The removal of one or more nucleotides from a nucleic acid molecule or one or more amino acids from a protein, where 3′ and 5′ ends of the nucleotide sequence may be removed, or the carboxy (C) and amino (N) terminal ends of the protein sequence removed and the nucleotide ends and/or amino/carboxy ends are subsequently re-ligated. A deletion can also refer to the removal of an N- or C-terminal segment, or a 3′ or 5′ terminal end of a nucleotide sequence, wherein the translated or transcribed products are shorter in sequence length than the starting sequence.

Detectable: Capable of having an existence or presence ascertained using various analytical methods as described throughout the description or otherwise known to a person skilled in the art.

Direct and indirect: As used herein, direct and indirect, in reference to exogenous control of a control element, refers to the effectuation of a genetic regulatory and/or expression change by the control element in response to the presence or absence of the exogenous compound, without which such a genetic regulatory and/or expression change would not occur. For example, the exogenous compound may interact directly with the control element to effect a change in the regulatory control by the control element. Alternatively, for example, the control element may indirectly effect a change in the regulatory control by the control element by interacting with one or more other cellular components that, in turn, can directly affect a change in the regulatory control by the control element.

DNA: Deoxyribonucleic acid. DNA is a long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached.

Down-regulation: Refers to when a gene is caused to be transcribed at a reduced rate compared to the endogenous gene transcription rate for that gene. In some examples, down-regulation additionally includes a reduced level of translation of the gene compared to the endogenous translation rate for that gene. Methods of testing for down-regulation are well known to those in the art. For example, the transcribed RNA levels can be assessed using RT-PCR, and protein levels can be assessed using SDS-PAGE analysis.

Downstream: Downstream, when describing a metabolic process, refers to the thermodynamically favored, in vivo enzymatic process of converting a substrate to a product, wherein the product can be, in turn, terminal or the substrate for another enzymatic process. While enzymatic processes are generally reversible, in a living cell a thermodynamic and/or kinetic directionality is preferred for an enzyme converting a substrate to a product, and is generally well known to the person having ordinary skill in the art. Downstream, when describing a metabolic process in a cell, can also refer to the enzymatic process of metabolizing (by catabolic or anabolic processes) a product or precursor compound from a substrate, for which the precursor compound can subsequently be the substrate for additional enzymatic reactions, a component of a molecular assembly, or a desired end-product. Downstream, when used in the description of a series of enzymatic reactions without a defined beginning or end, for example the citric acid cycle, refers to the natural thermodynamic or kinetic direction of a chemical reaction, whether anabolic or catabolic, for product formation dependant on the physiological state of the cell. Downstream, when describing the location of a nucleic acid sequence, refers to 1) the nucleic acid sequence 3′ to a nucleic acid sequence described, and/or 2) the translation, transcription, regulation or other related activity performed on a second nucleic acid sequence occurring after the translation, transcription, regulation or other related activity performed on a first nucleic acid sequence.

Endogenous: As used herein with reference to a nucleic acid molecule and a particular cell or microorganism, refers to a nucleic acid sequence or peptide that is in the cell and was not introduced into the cell (or its progenitors) using recombinant engineering techniques. For example, a gene that was present in the cell when the cell was originally isolated from nature. A gene is still considered endogenous if the control sequences, such as a promoter or enhancer sequences that activate transcription or translation, have been altered through recombinant techniques.

Enzyme activity: As used herein, the term an “enzyme activity” refers to an indicated enzyme (e.g., an “alcohol dehydrogenase activity”) having measurable attributes in terms of, e.g., substrate specific activity, pH and temperature optima, and other standard measures of enzyme activity as the activity encoded by a reference enzyme (e.g., alcohol dehydrogenase). Furthermore, the enzyme is at least 90% identical at a nucleic or amino acid level to the sequence of the reference enzyme as measured by a BLAST search.

Enzyme Classification Numbers (EC): The EC numbers provided throughout this description are derived from the KEGG Ligand database, maintained by the Kyoto Encyclopedia of Genes and Genomics, sponsored in part by the University of Tokyo. The EC numbers are as provided in the database on Feb. 1, 2008.

Exogenous: As used herein with reference to a nucleic acid molecule and a particular cell or microorganism, exogenous refers to a nucleic acid sequence or peptide that was not present in the cell when the cell was originally isolated from nature. For example, a nucleic acid that originated in a different microorganism or synthesized de novo and was engineered into an alternate cell using recombinant DNA techniques or other methods for delivering said nucleic acid is exogenous. Exogenous with reference to a compound or organic compound refers to an extracellular compound or organic compound required for the growth, propagation, sustenance, viability or activity of any metabolic activity, without specific reference to any one metabolic activity. The exogenous compound or organic compound includes those that are subsequently converted by the microorganism to metabolites and/or intermediates necessary or useful for cellular function.

Exponential growth and linear growth: Exponential growth (or exponential population density or exponential population growth) refers to the exponential increase in cell density in cell cultures resulting from the doubling of cells per unit time period. As used herein, as long as the OD₇₃₀ of the culture is below some relatively low value, for example, OD₇₃₀ of approximately 0.7 for a 30 ml culture in a 125 ml flask in a shaking photoincubator set to approximately 100 μmol photons m⁻² s⁻¹, and neither CO₂ nor inorganic nutrients are limiting and, as long as the culture is not limited in the amount of photon flux/lighting available, there is an exponential phase of cell amplification, akin to that commonly observed for heterotrophic bacteria such as E. coli grown in Luria broth. Under such conditions, the cyanobacteria grow at an exponentially increasing growth rate, denoted by μ with units of inverse time, that is equal to ln(2)/γ where γ is the exponential-phase doubling time. Linear growth (or linear population density or linear population growth), as used herein, refers to a linear population expansion (occurring from an OD₇₃₀ of approximately 0.80 to >40.0) following an initial exponential expansion of population density (occurring from an OD₇₃₀ of approximately 0.0 to 0.8) before reaching the stationary phase wherein little or no population growth expansion is observed. Once the OD₇₃₀ of the culture reaches a value beyond which essentially all the incident photosynthetically active radiation (PAR) is absorbed, and, therefore, once the culture first becomes light limited, there begins a linear, light-limited, phase of cell amplification. Under such conditions, the strain grows at a constant, linear growth rate, denoted by m with units of cell concentration per unit time, that is equal to (C₂−C₁)/(t₂−t₁), where C₁ equals the cell concentration at time t₁, and C₂ equals relatively greater cell concentration at a later time t₂. Analogous to the exponential phase doubling time γ, the linear phase doubling time g can be calculated using values of C₂, C₁, t₂, and t₁ such that at t₂, C₂=2*C₁. As expected of the different degrees of light limitation of linear and exponential growth regimes, g is almost always significantly larger than γ. The exponential and linear phases do not always follow precisely mathematically exponential or linear functions. For example, it is not uncommon to observe that, upon the initiation of light limitation, the growth rate of certain cyanobacterial strains (especially highly productive metabolically engineered hosts that divert, at the expense of biomass, a majority of their fixed carbon into one or a small number of desired end-products) progressively and smoothly decreases over time up to entry into stationary phase, rather than being constant as would be expected of precise linear growth.

Expression: The process by which nucleic acid encoded information of a gene is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Expression Control Sequence: as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

Fixed Nitrogen Source: A fixed nitrogen source refers to any form of soluble nitrogen metabolically active in a microorganism, including, but not limited to, salts of nitrogen (for example, NH₃, ammonium salts, amides, imides, nitrides) and oxides of nitrogen (for example nitrates and nitrites).

Fuel component: refers to any compound or a mixture of compounds that are used to formulate a fuel composition. There are “major fuel components” and “minor fuel components.” A major fuel component is present in a fuel composition by at least 50% by volume; and a minor fuel component is present in a fuel composition by less than 50%. Fuel additives are minor fuel components. The isoprenoid compounds disclosed herein can be a major component or a minor component, by themselves or in a mixture with other fuel components.

Genetic element: A genetic element refers to any functional, regulatory or structural nucleic acid or nucleic acid sequence such as, without limitation, ribonucleic acid and deoxyribonucleic acid (RNA, DNA), whether originating from exogenous or endogenous sources, derived synthetically or originating from any organism or virus, including, without limitation, cDNA, genomic DNA, mRNA, RNAi, snRNA, siRNA, miRNA, ta-siRNA, tRNA, double stranded and/or single stranded, co-suppression molecules, ribozyme molecules or related nucleic acid constructs.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Immiscible or Immiscibility: refers to the relative inability of a compound to dissolve in water and is defined by the partition coefficient “P” of the compound. The partition coefficient, P, is defined as the equilibrium concentration of compound in an organic phase (in a bi-phasic system the organic phase is usually the phase formed by the fatty acid derivative during the production process, however, in some examples an organic phase can be provided (such as a layer of octane to facilitate product separation) divided by the concentration at equilibrium in an aqueous phase (i.e., fermentation broth). When describing a two phase system the P is usually discussed in terms of log P. A compound with a log P of 10 would partition 10:1 to the organic phase, while a compound of log P of 0.1 would partition 10:1 to the aqueous phase.

Isolated: An “isolated” nucleic acid or polynucleotide (e.g., RNA, DNA or a mixed polymer) refers to one which is substantially separated from other cellular components that naturally accompany the native polynucleotide in its natural host cell, e.g., ribosomes, polymerases, and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems. However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g. by homologous recombination) for the native promoter of a gene in the genome of a human cell, such that this gene has an altered expression pattern. This gene would now become “isolated” because it is separated from at least some of the sequences that naturally flank it. A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g. by human intervention. An “isolated nucleic acid” also includes a nucleic acid integrated into a host cell chromosome at a heterologous site, as well as a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized. The term also embraces nucleic acid molecules and proteins prepared by recombinant expression in a host cell as well as chemically synthesized nucleic acid molecules and proteins.

Knock-out: Refers to a gene whose level of expression or activity has been reduced to zero. In some examples, a gene may be knocked-out with deletion of some or all of its coding sequence. In other examples, a gene may be knocked-out with an introduction of one or more nucleotides into its open-reading frame, which can result in translation of a non-sense or otherwise non-functional protein product.

Metabolic junction: A metabolic junction refers to a substrate(s) and the enzymatic (metabolic) pathways that compete for the substrate(s) for conversion of the substrate into a product or biomass. A metabolic junction comprises at least two enzymes, and can comprise three or more enzymes, from different metabolic pathways, that recognize the same or similar substrates. The competing metabolic pathways are divergent, converting the substrate into: a different chemical product that may be an end-product, a metabolite to be converted into other products through additional metabolic pathways, or biomass.

Nucleic acid molecule: Nucleic acid molecule refers to both RNA and DNA molecules including, without limitation, cDNA, genomic DNA and mRNA, and also includes synthetic nucleic acid molecules, such as those that are chemically synthesized or recombinantly produced. The nucleic acid molecule can be double-stranded or single-stranded, circular or linear. If single-stranded, the nucleic acid molecule can be the sense strand or the antisense strand.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. Configurations of separate genes that are transcribed in tandem as a single messenger RNA are denoted as operons. Thus placing genes in close proximity, for example in a plasmid vector, under the transcriptional regulation of a single promoter, constitutes a synthetic operon.

Overexpression: When a gene is caused to be transcribed at an elevated rate compared to the endogenous transcription rate for that gene. In some examples, overexpression additionally includes an elevated rate of translation of the gene compared to the endogenous translation rate for that gene. Methods of testing for overexpression are well known in the art, for example transcribed RNA levels can be assessed using reverse transcriptase polymerase chain reaction (RT-PCR) and protein levels can be assessed using sodium dodecyl sulfate polyacrylamide gel elecrophoresis (SDS-PAGE) analysis. Furthermore, a gene is considered to be overexpressed when it exhibits elevated activity compared to its endogenous activity, which may occur, for example, through reduction in concentration or activity of its inhibitor, or via expression of mutant version with elevated activity. In preferred embodiments, when the host cell encodes an endogenous gene with a desired biochemical activity, it is useful to over-express an exogenous gene, which allows for more explicit regulatory control in the fermentation and a means to potentially mitigate the effects of central metabolism regulation, which is focused around the native genes explicitly.

Productivity: Productivity, as used herein, is the rate at which carbon-based products are produced by a host cell, and is generally expressed in grams or millgrams/liter/hour((gm)(mg)/L/hr). Productivity of a host cell is not constant; for example, productivity can vary depending on if the host cell is in a stationary phase of growth, a linear stage of growth, an exponential stage of growth, or transitions between growth stages. Productivity in a photoautotroph is related to many factors including temperature, bioreactor construction and design (where bioreactors are used), photosynthetic photon flux density, exposed surface area of growth media, optical density of growth media, cell density, specific host cell strain, specific carbon-based product synthesized, and the like. Thus, a comparison of productivity among host cell strains, growth conditions, bioreactor types, etc., generally cannot be meaningfully made without a measure for photosynthetic efficiency. Productivity measures as used herein refer generally to production strains with and without the invention described herein, cultured in identical or nearly identical conditions and parameters.

Purified: The term purified does not require absolute purity; rather, it is intended as a relative term. Thus, for example, a purified product preparation, is one in which the product is more concentrated than the product is in its environment within a cell. For example, a purified wax is one that is substantially separated from cellular components (nucleic acids, lipids, carbohydrates, and other peptides) that can accompany it. In another example, a purified wax preparation is one in which the wax is substantially free from contaminants, such as those that might be present following fermentation.

Recombinant: A recombinant nucleic acid molecule or protein is one that has a sequence that is not naturally occurring, has a sequence that is made by an artificial combination of two otherwise separated segments of sequence, or both. This artificial combination can be achieved, for example, by chemical synthesis or by the artificial manipulation of isolated segments of nucleic acid molecules or proteins, such as genetic engineering techniques. Recombinant is also used to describe nucleic acid molecules that have been artificially manipulated, but contain the same regulatory sequences and coding regions that are found in the organism from which the nucleic acid was isolated.

The terms “recombinant host cell” (“expression host cell,” “expression host system,” “expression system,” or simply “host cell” or “strain”), as used herein, refers to a cell into which a recombinant vector has been introduced, e.g., a vector comprising acyl-CoA synthase. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

Release: The movement of a compound from inside a cell (intracellular) to outside a cell (extracellular). The movement can be active or passive. When release is active it can be facilitated by one or more transporter peptides and in some examples it can consume energy. When release is passive, it can be through diffusion through the membrane and can be facilitated by continually collecting the desired compound from the extracellular environment, thus promoting further diffusion. Release of a compound can also be accomplished by lysing a cell.

Substantially pure: As used herein, a composition that is a “substantially pure” compound is substantially free of one or more other compounds, i.e., the composition contains greater than 80 vol. %, greater than 90 vol. %, greater than 95 vol. %, greater than 96 vol. %, greater than 97 vol. %, greater than 98 vol. %, greater than 99 vol. %, greater than 99.5 vol. %, greater than 99.6 vol. %, greater than 99.7 vol. %, greater than 99.8 vol. %, or greater than 99.9 vol. % of the compound; or less than 20 vol. %, less than 10 vol. %, less than 5 vol. %, less than 3 vol. %, less than 1 vol. %, less than 0.5 vol. %, less than 0.1 vol. %, or less than 0.01 vol. % of the one or more other compounds, based on the total volume of the composition.

Suitable fermentation conditions. The term generally refers to fermentation media and conditions adjustable with, pH, temperature, levels of aeration, etc., preferably optimum conditions that allow microorganisms to produce carbon-based products of interest. To determine if culture conditions permit product production, the microorganism can be cultured for about 24 hours to one week after inoculation and a sample can be obtained and analyzed. The cells in the sample or the medium in which the cells are grown are tested for the presence of the desired product.

Vector: The term “vector” as used herein refers to a nucleic acid molecule capable of transporting another nucleic acid to which it has been linked. One type of vector is a “plasmid,” which refers to a circular double-stranded DNA loop into which additional DNA segments may be ligated. Other vectors include cosmids, bacterial artificial chromosomes (BACs) and yeast artificial chromosomes (YACs). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”). A vector can also include one or more selectable marker genes and other genetic elements known in the art.

### Metabolic Switches

The invention described herein identifies pathways, mechanisms and methods to confer a capacity to switch effectively between biomass production and the production of ethanol, fatty acid derivatives and/or other carbon-based products of interest directly to host microorganisms, including, without limitation, photoheterotrophs, chemoheterotrophs and photoautotrophs. Photoautotrophs engineered in this capacity are uniquely enabled in the efficient production of carbon-based products of interest directly from carbon dioxide and light, eliminating the time-consuming and expensive processing steps currently required to generate biofuels and biochemicals from biomass sources such as corn, sugar cane, miscanthus, cellulose, and others. Accordingly, the microorganisms of the invention are capable of synthesizing effectively and releasing ethanol, fatty acid derivatives and carbon-based products derived from various biosynthetic pathways by fixing CO₂.

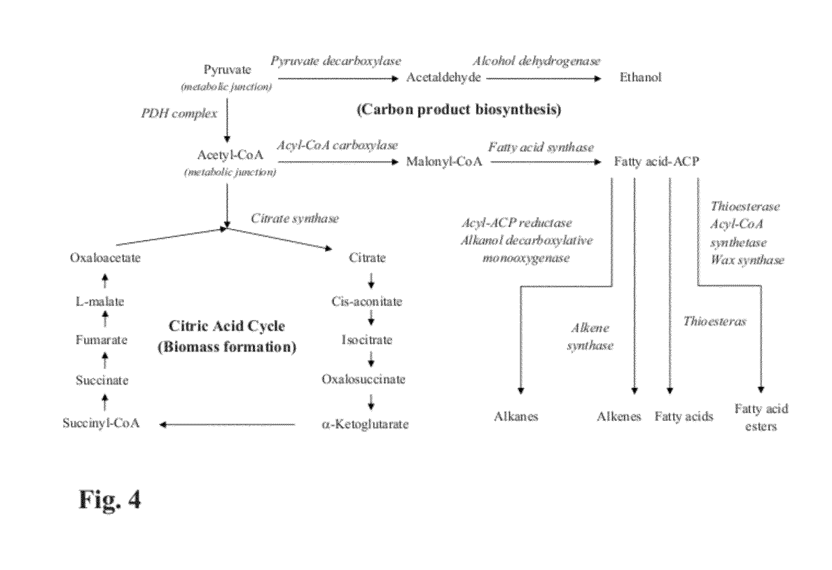

A “metabolic switch” of the present invention generally refers to the combination of 1) a host organism auxotrophic for an exogenous compound, 2) the exogenous compound indirectly or directly acting upon a control element (a genetic element or protein) 3), the control element attenuating or activating, directly or indirectly, carbon flux into two or more metabolic pathways, 4) the metabolic pathways diverging from a shared metabolic junction using a shared initial substrate to different chemical intermediates or products, and 5) the shared initial substrate being directed to a particular pathway to the substantial or complete exclusion of the other(s) pathways responsive to the control element. FIG. 4. It is preferred that a control element regulates the carbon flux at a metabolic junction wherein biomass is produced downstream of or as a product of at least one exiting metabolic pathway, and desirable carbon-based products are produced downstream of or as a product of at least one other exiting metabolic pathway. Additionally, a metabolic junction is chosen wherein the common substrate has limited, or can be engineered to have limited, cataplerotic metabolic pathways in which the substrate would otherwise be subjected to unwanted chemical conversion or transformation. A selected metabolic junction that has few or no cataplerotic pathways with which it can circumvent the metabolic switch therefore will minimally dissipate the common substrate pool. Rather, carbon flux preferably is forced through one of the desired pathways, for example resulting in an increase of the total amount of either biomass or end-product biosynthesis. FIG. 4.

Metabolic switches engineered into microorganisms for the present invention have several advantages over common inducible genetic systems. Metabolic switches allow carbon flux through one metabolic pathway to be substantially or completely diverted to another pathway, mitigating loss of carbon flux to competing cataplerotic metabolic pathways. In effect, with a metabolic switch, carbon is being diverted from one pathway directly to another rather than being partitioned to several pathways and a lower net efficiency for any one particular end-product synthesis or biomass production. Therefore, control over the activation of metabolic pathways with a metabolic switch allows for dedication of cellular carbon flux to singular specific and desired metabolic purposes or synthesis of end-products (for example, either ethanol or biomass production). Furthermore, with carbon flux diversion or re-direction, common issues existing in other expression systems (including unwanted low-level background expression) are avoided, adding to the net productive efficiency of biosynthetic end-products.

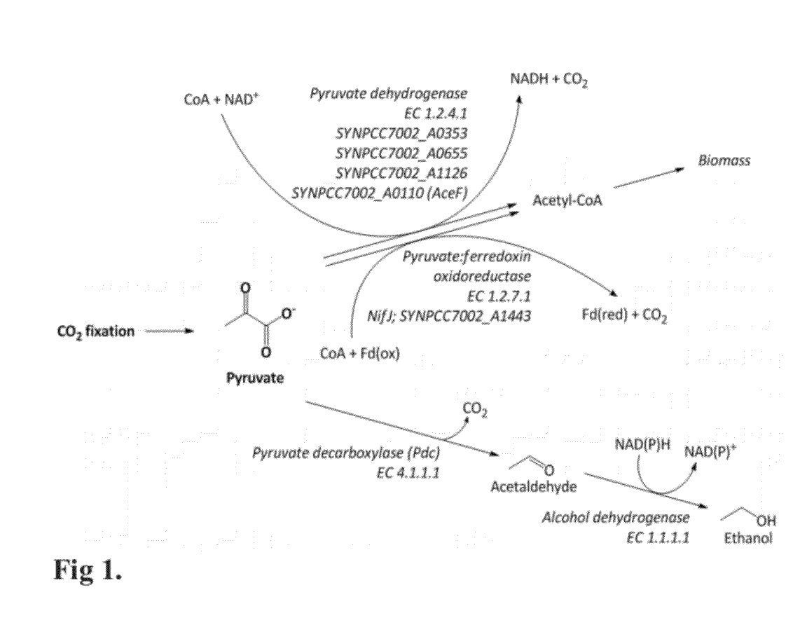

When controlling cellular processes at metabolic junctions, a single metabolic switch can allow for the regulation of multiple biosynthetic pathways. For example, pyruvate can be directed by the cell towards the synthesis of acetyl-CoA, lactate formation, amino acid formation, and (with heterologous genes of the current invention) ethanol formation. Therefore, pyruvate sits at a metabolic junction wherein it is used as a substrate for these alternative metabolic pathways. FIG. 4. In this invention, in one embodiment pyruvate metabolism is directed by exogenously supplied lipoic acid. Lipoic acid activates the pyruvate dehydrogenase complex known to require the lipoylation of AceF (the E2 dihydrolipoamide acetyltransferase subunit). Therefore, the metabolic switch, employing AceF as the control element, directs carbon flux into or away from pyruvate dehydrogenase metabolism at the pyruvate metabolic junction. The lipoic acid activated complex then directs pyruvate flow to acetyl-CoA or, alternatively, when deactivated, forces pyruvate through a metabolic pathway for ethanol biosynthesis, and potentially, other cataplerotic pathways. FIG. 1. Thus, as opposed to inducible expression systems being “on” or “off,” metabolic switches confer control over cellular carbon flux with “either/or” activity by channeling carbon flux through one metabolic pathway while inactivating other pathway(s) extending from the junction.

In contrast with common expression systems, metabolic switches can control carbon flux throughput in complete metabolic pathways rather than the activity of only one or a few induced gene products. Therefore, metabolic switches are advantageous in that they can alternate and/or control the activity level between or among complete metabolic pathways.

It has long been sought to have a system wherein biomass production of a host organism can be attenuated without adversely affecting the biosynthetic productivity of other desired metabolic pathways. This invention describes a metabolic switch controlling carbon flux wherein host organisms can be grown efficiently to any desired population density, then activating (or deactivating) a control element at which point the carbon flux is channeled to biosynthesis of desired carbon-based products including ethanol.

### Engineered Microorganisms Auxotrophic for Exogenous Compounds

Described herein are embodiments of a metabolic switch used to divert cellular carbon from use in biomass production to production of ethanol, fatty acid derivatives and other carbon-based products. Host cells are engineered to be auxotrophic for an exogenous compound that controls the activity of both biomass-producing metabolic pathways and heterologous metabolic pathways sharing a metabolic junction. Treatment of host cell growth media with the exogenous compound activates at least one metabolic pathway and deactivates at least one other. Removal of the exogenous compound reverses the activity.

Industrially important engineered organisms in some instances produce greater quantities of desirable products during exponential growth while dropping productivity levels during a stationary phase (Brik Ternbach, M. et al., (2005) Biotechnol. Bioeng. 91:356-368; Elis\check{}a'kova', V., et al. (2005). Appl. Environ. Microbiol. 71:207-213. Huser, A., et al., (2005). Appl. Environ. Microbiol. 71:3255-3268). Thus, productivity and yield of important biosynthetic compounds may intrinsically be linked to active biomass production metabolic pathways. In such cases, productivity and yield may decrease when host cells enter a stationary phase because biomass-producing metabolic pathways become inactive. Inactivation of the biomass producing pathways can be caused by quorum sensing, toxic buildup in growth media, nutrient deprivation, oxidative damage of cellular components, culture cell density impeding photon penetration (in the case of photosynthetic organisms), and other effects of host cell population density, with secondary effects manifested as global genetic attenuation, including biosynthetic pathways. Therefore, it is desirable to effect exogenous control over the biomass-producing metabolic pathways to attenuate host cell growth prior to the population entering the stationary phase. In so doing, biosynthetic pathways maintain productivity at optimal cell densities (and can produce high yields) wherein the host cells remain in a quasi-linear growth metabolic state. Alternatively, the metabolic switch can toggle between activation of metabolic pathways for biomass growth during which vital cellular repair, regeneration of replenishment activity occurs and metabolic pathways for carbon-based product biosynthesis. Thus, productivity and yield of carbon-based products of interest can be improved substantially over those observed decreased values occurring in the stationary phase.

Host cells can be evaluated for productivity and yield to determine if such genetic alterations result in improved host strains. Volumetric productivity, “V₂,” (in mass of product/liter/hour) of a host cell is evaluated with and without a metabolic switch during both the linear growth stage and stationary phase by sampling the output of a desired product at various time intervals. Biomass, “V₃,” in terms of dry cell weight, is evaluated by methods well known to those persons with skill in the art (vide infra). Thus, overall product yield, “Y,” can be cast in terms of eq. 1:

V₂/(V₂+V₃)=Y Eqn. 1

where Y is the percentage of product yield out of the total biomass and carbon-based product produced by the cell.

During exponential and linear growth, carbon flux is partitioned among biomass growth, essential cataplerotic pathways and (some) biosynthesis of product of interest. In stationary phase, carbon flux to biomass production becomes attenuated, and it is desirable that carbon flux used for biomass be diverted to biosynthesis of product. If carbon diversion occurs with no other significant cellular changes, it is expected that productivity levels for carbon products will remain constant or even increase. However, this effect is not always observed because of secondary effects (for example, quorum sensing, growth medium toxicity from cellular effluvium, oxidative damage, nutrient/photon limitation) that attenuate global gene expression (in addition to biomass and heterologous genes), yet keep activated genes involved in cellular maintenance. Engineering a metabolic switch into the host cell allows for the complete control over diversion of cellular carbon from biomass production to biosynthesis of carbon-based products, and can be initiated at any time/density point in population growth. Therefore, partitioning of cellular carbon to alternate metabolic pathways is decreased and pools of carbon metabolites are increasingly available for specific product formation. Thus, if the metabolic switch is activated at an optimal cell density within the linear growth phase, one of three effects is expected: 1) productivity (V₂) remains at levels present during linear growth through the productive lifetime of the cell culture, 2) productivity increases because nutrients such as cellular carbon that are channeled to biomass production would become available for biosynthesis of carbon-based products (V₂ increases and V₃ decreases), or 3) productivity (V₂) increases because photon flux is directed towards producing cellular carbon that can be channeled directly to biosynthesis of carbon-based products. This latter effect introduces a fourth term for the energetic efficiency of biosynthesis (“η_{product}”) for a carbon-based product, and can be defined as the ratio of V₂ to photon flux used during the total biosynthesis of the carbon-based product. Both photon flux and V₂ are evaluated for total energy in Joules by methods described herein (vide infra). Thus, η_{product} product allows for a normalization of evaluation parameters and sets forth a universal measure for how efficient a host cell synthesizes a product regardless of the specific photobioreactor technology used.

Important aspects of the invention include attenuation of the production of biomass (and concomitant increase in ethanol production) and fatty acid derivatives by controlling Pdh and/or citrate synthase activity directly. Also important for the present invention is the attenuation of cataplerotic pathways, for example attenuating pyruvate:ferredoxin oxidoreductase, pyruvate formate lyase and lactate dehydrogenase to eliminate these alternative pathways of pyruvate metabolism to acetyl-CoA conversion and other metabolites. This is achieved by genetic, expression, primary amino acid sequence and/or structural modifications to, without limitation, aceF, lplA, ldhA, pdh, aceE, nig, ldhA, pps, and/or homologous genes encoding dihydrolipoyl transacetylase, pyruvate dehydrogenase and dihydrolipoyl dehydrogenase.

In preferred aspects, the exogenous compound is lipoic acid or a fixed nitrogen source. Upon provision or removal of an exogenous compound, carbon flux through a metabolic pathway is diverted from one or more pathways to other desired metabolic pathways. For example, in embodiments described (infra) engineered with such a metabolic switch, by employing an exogenous compound, pyruvate can be diverted from its use in biomass and fatty acid derivatives production to instead be used for ethanol production. FIG. 4. Specifically, depletion of the exogenous compound results in the channeling of cellular carbon either to the production of ethanol or to the production of biomass/fatty acid derivatives. FIG. 1. Similarly, other embodiments of the present invention disclose the use of a metabolic switch to regulate the carbon flux through acetyl-CoA to biomass/TCA cycle production or biosynthesis of fatty acid derivatives. FIG. 4. These metabolic switches feature the use of lipoic acid and a fixed nitrogen source as exogenously supplied compounds for host cells auxotrophic for these compounds.

In various embodiments, the host cells comprise a heterologous pyruvate decarboxylase activity selected from Zymobacter palmae and Zymomonas mobilis Pdc. In various other embodiments, the host cells comprise a heterologous alcohol dehydrogenase activity selected from Z. mobilis adhII, Z. mobilis AdhII TS42 and Z. mobilis AdhB. In other related embodiments, the host cells comprise a NADPH-dependent alcohol dehydrogenase activity. In another embodiment, the NADPH-dependent alcohol dehydrogenase activity is heterologous Moorella sp. HUC22-1 AdhA. In yet another embodiment, host cells synthesize fatty acid derivatives including, but not limited to, alkanes (by which U.S. Pat. No. 7,794,969 is incorporated by reference in its entirety).

In one particular embodiment, a host cell is auxotrophic for lipoic acid. The lipoic acid auxotroph is engineered for attenuation of endogenous pathways leading to lipoylation of aceF gene product dihydrolipoyl transacetylase (where the Pdh E2 subunit dihydrolipoyl transacetylase is the control element) and has the AceF protein control element controlled by exogenously supplied lipoic acid. Additionally, the lipoic acid auxotroph is engineered to have a deletion for a gene encoding lipoyl synthase (LipA) and additions of genes encoding Pdc, Adh and Lipoyl protein ligase (LplA; E.C #2.7.7.63) (ΔlipA pdc adh lpl). Attenuation or deletion of LipA renders the host cell dependent on lipoic acid. Therefore, when presented to the host cell exogenously, lipoic acid diffuses or is actively transported into the cell where heterologous LplA protein ligates lipoic acid onto the dihydrolipoyl transacetylase subunit of Pdh. Related embodiments include host cells attenuated for acyl-ACP synthetase and/or LipB activity.

In other various aspects, the invention provides a composition and method wherein a host cell has a heterologous lipoic acid membrane transport pathway (panF, yipK, SMVT, lplA, lipT, ecfA1, ecfA2, efcT), heterologous Pdc and Adh activity, attenuated endogenous lipoic acid synthesis and/or metabolism pathways (for example, and without limitation, aas1, aas2, lipB, lipA1, lipA2), and the capacity for regulating the host cell with the exogenous compound lipoic acid for biomass growth or the generation of carbon-based products.

In a second particular embodiment, a host cell is auxotrophic for a fixed nitrogen source, expresses lipoamidase protein or variants thereof with a Pₙᵢᵣ inducer sequence as the control element and the Pₙᵢᵣ control element indirectly controlled by exogenously supplied nitrate. The host cell is engineered to have a functional lipoamidase gene, wherein nitrate as a cellular fixed-nitrogen resource directs the expression of lipoamidase to attenuate Pdh activity. Upon Pdh attenuation, cellular pyruvate levels accumulate for conversion to ethanol through the activity of heterologous expressed Pdc and Adh. In the presence of exogenous nitrate, the nitrate reduction pathway is activated. An intermediate product of this pathway, nitrite, will activate expression of endogenous and/or heterologous Pₙᵢᵣ promoters specifically activated in the presence of nitrite. In alternative embodiment, a Pₙᵢᵣ promoter is engineered to express both lipoamidase and genetic elements for ethanol production. Lipoamidase enzyme will deactivate Pdh, thereby preventing pyruvate from being used for biomass through the acetyl-coA intermediate, and instead pyruvate will be channeled to ethanol production.

In a third particular embodiment of the present invention, a host cell is auxotrophic for a fixed nitrogen source, expresses endogenous or heterologous citrate synthase protein with a Pₙᵢᵣ inducer sequence as the control element and has the Pₙᵢᵣ control element indirectly controlled by exogenously supplied nitrate. The host cell is engineered to have a functional citrate synthase gene, wherein conversion of nitrate as a cellular fixed-nitrogen resource directs the expression of citrate synthase which converts acetyl-CoA to citrate. Upon production of citrate, the TCA cycle is active and leads to the production of biomass. When nitrate is replace by urea, Pₙᵢᵣ is inactive and citrate synthase is no longer produced to convert acetyl-CoA to citrate. Acetyl-CoA pools can build up to supply metabolic pathways for the biosynthesis of fatty acid derivatives and carbon-based products of interest.

In a fourth particular embodiment, a host cell is auxotrophic for two exogenous compounds. For example, the host cell is auxotrophic for a fixed nitrogen source to activate Pₙᵢᵣ controlled synthesis of citrate synthase and lipoic acid to control Pdh activity. Therefore, a single host cell is capable of producing ethanol, fatty acid derivatives or biomass based upon the supply of exogenous fixed nitrogen sources and lipoic acid.

In embodiments of the present invention, controlling pyruvate conversion to other biomolecules requires the inactivation, deactivation or specific control over pyruvate dehydrogenase. Therefore, if other pathways were available for metabolic oxidation of pyruvate to acetyl-coA, the purpose of the invention would be subverted. Enzyme pathways converting pyruvate to other metabolites are therefore attenuated in order to maintain elevated pyruvated pool concentrations. For example, lactate dehydrogenase activity (EC 1.1.1.27) is eliminated with a deletion of a functional ldhA gene, and phosphoenolpyruvate synthase activity (EC 2.7.9.2) for the conversion of pyruvate to phosphoenolpyruvate is eliminated with a deletion of a functional pps gene. To that end, for the purpose and intent of the present invention, other enzyme activities non-vital for a viable host cell using pyruvate as a substrate are also disabled, attenuated or otherwise rendered non-functional, and include but are not limited to pyruvate formate lyase (EC 2.3.1.54) and pyruvate ferredoxin:oxidoreductase (EC 1.2.7.1).

### Lipoic Acid Directed Metabolic Switches

Various aspects of the invention provide for compositions of genetically engineered bacterial strains auxotrophic for lipoic acid (lipoate) that, upon depletion of lipoate, minimize biomass production and maximize ethanol production. This conversion between optimal biomass or ethanol production occurs by channeling pyruvate from the biomass producing, endogenous pyruvate dehydrogenous metabolic pathway to the ethanol generating, heterologous pyruvate decarboxylase/alcohol dehydrogenase (Pdc/Adh) pathway. By altering the activity of the pyruvate dehydrogenase complex, upon which the cell is made to depend completely for conversion of pyruvate to acetyl-CoA, the switch between biomass production and ethanol biosynthesis is achieved. FIG. 1.

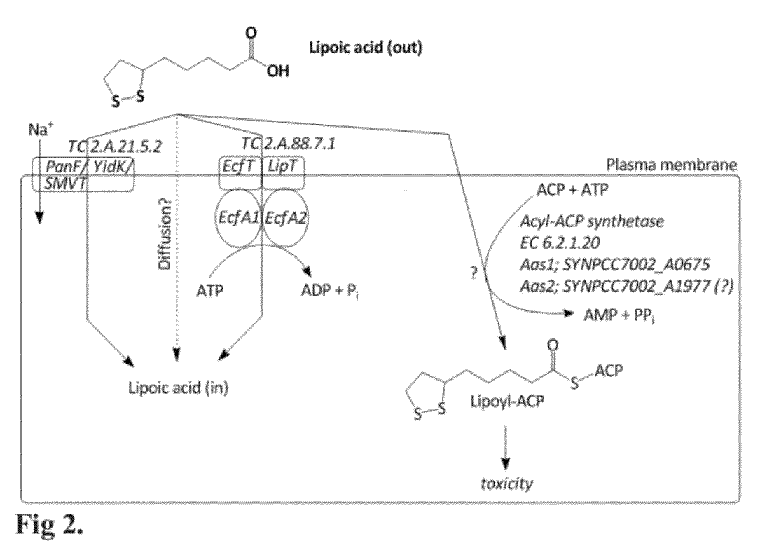

The enzymatic activity of the Pdh complex is itself dependent on being activated by the dihydrolipoamide acetyltransferase protein (AceF, the E2 component of the Pdh complex). FIG. 2. Therefore, control of Pdh activity can be achieved through controlling the AceF protein. A novel aspect of the invention is to regulate the function of Pdh by controlling the activity of this essential Pdh component, AceF, in the context of a metabolic switch. Cells are grown to the desired density by supplying the minimal amount lipoic acid for AceF to activate the Pdh complex, with carbon flux being diverted to biomass formation rather than ethanol. When lipoic acid is depleted, Pdh becomes inactive, cell populations no longer increase in biomass, pyruvate accumulates and ethanologensis preferentially proceeds utilizing the Pdc-Adh pathway. Thus, a novel aspect of this invention is the rational re-direction of pyruvate carbon flux from biomass production to ethanol biosynthesis by altering the exogenous compound composition (i.e., lipoate concentration) of the growth medium.

Exogenous control of the host cell using lipoic acid further requires attenuation of alternate lipoate metabolic pathways. FIG. 2. In one embodiment, a host cell auxotrophic for lipoate (lipoic acid) is engineered so that the endogenous acyl-ACP synthetases no longer activate lipoic acid through the acyl-ACP pool. More specifically, one embodiment of the invention is to prevent the cell from channeling exogenous lipoic acid into the acyl-ACP pool via acyl-ACP synthetase (Aas1 and or Aas2). Acyl-ACP synthetase functions to channel exogenous fatty acids into the cell as well as recycle those fatty acids found endogenously. However, if lipoic acid enters the acyl-ACP pool, potentially the lipoate molecule can be elongated and incorporated into membranes as a toxin or generally be inhibitory to further processing of the acyl-ACP pool. Therefore, acyl-ACP synthetases (EC 6.2.1.20; aas1 and/or aas2) and their homologues are deleted from the genome, genetically altered so translation and/or transcription does not occur, or are genetically altered so that if expression occurs, the protein product is non-functional.

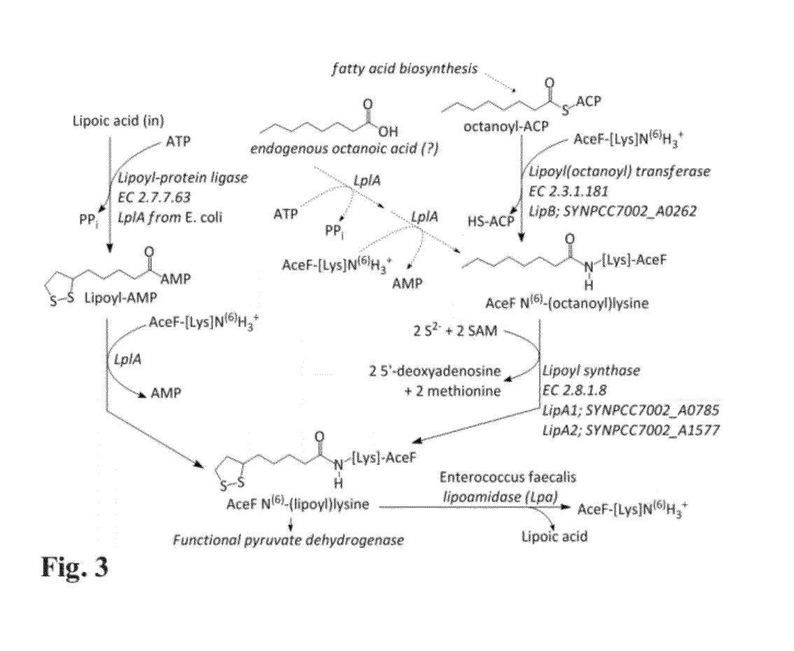

In another embodiment, a cell auxotrophic for lipoate is engineered so that the transport of lipoate across the membrane is enhanced above intrinsic diffusion rates, increasing the rate of transfer or overall flux of lipoate molecules into the cell. Aside from or in combination with the increased transmembrane transport rate, active transport can result in increased cellular concentrations of lipoate using lower concentrations of exogenous lipoate during, for example, an industrial process. FIG. 3. Specifically, one embodiment is to genetically engineer the lipoate auxotroph to express proteins of the sodium:solute symporter family to facilitate increased amounts of exogenous lipoate to be transferred into the cell. FIG. 3. Specifically, mammalian sodium dependent multivitamin transporter (SMVT; NCBI Accession #AAC64061) is capable of transporting lipoate and other important biomolecules across the membranes, is useful for increasing lipoate transfer in a lipoate auxotroph, and therefore is engineered into the auxotroph. Another embodiment is to genetically engineer protein expression of genes homologous to known proteins of the sodium:solute symporter family, such as SMVT, for expression in the lipoate auxotroph. Bacterial homologous to SMVT include, but are not limited to, YidK (Accession #AAC76702) and PanF (Accession #AAA24276). In yet another embodiment, combinations of one or more homologues to SMVT, YidK and/or PanF are genetically engineered into a lipoate auxotroph to maximize transfer of lipoate into the cell. An alternative to using a sodium:solute symport transporter is to use a lipoate transport system of the energy-coupling factor family. FIG. 3. This system uses an endogenous soluble ATP-binding component and a transmembrane component functioning in concert with an integral membrane substrate-specific (S) component. The LipT protein (Accession #CAM11872) is predicted to be the S component binding exogenous lipoate. In one embodiment, LipT, in combination with other components of an energy-coupling factor family, such as EcfA1(Accession #CAM11510) and EcfA2 (Accession #CAM11509), both ATP binding protein components, and EfcT (Accession #CAM11508), a transmembrane protein component, a functional lipoate transmembrane transport is used to increase endogenous lipoate levels.

In certain embodiments, the host cell comprises attenuated endogenous lipoyl synthase activity by altering or deleting the LipA protein products involved in the conversion of octanoate from endogenous fatty acid biosynthesis to lipoate. FIG. 2. There are two such genes in Synechococcus sp. PCC7002, lipA1 and lipA2. Knockouts, repression or mutagenesis of these genes renders the cell dependent on exogenously available lipoic acid for growth, and Pdh activity can thus be controlled by varying the concentration of exogenous lipoic acid in the growth medium. BLAST analysis of cyanobacterium of interest show that lplA or its homologues, an essential gene for converting lipoate to an active, phosphorylated form and thereby allowing lipoylation to occur on AceF and homologues, may absent in the genome. Therefore, even though the lipoate auxotroph is capable of taking in exogenous lipoic acid as described supra, it is not expected to be able to lipoylate proteins in the AceF family in the absence of endogenous lipoylation biosynthesis provided by lipA1, lipA2 and lipB. Therefore, in one embodiment of the present invention, the lplA gene of E. coli is engineered into the lipoate photoautotrophic auxotroph. FIG. 2. The gene product of lplA (EC 2.7.7.63) activates AceF proteins and homologues having a close consensus sequence to the lipoylation site of the lipoate AceF protein of interest. In another embodiment, heterologous genes and their homologues encoding for lipoyl (octanoyl) transferase (EC 2.3.1.181) and lipoyl synthase (EC 2.8.1.8) are genetically engineered into the lipoate auxotroph described herein to activate the endogenous AceF protein.

In some instances, endogenous octanoic acid can serve as a substrate for LplA, resulting in LplA-octanoylated AceF. FIG. 2. Similarly, endogenous LipB protein of the lipoate auxotroph can octanoylate AceF, and both LplA and LipB mediated octanoylation results in the production of an unnecessary cellular resource. Therefore, to decrease carbon flux through unwanted biosynthetic pathways, the lipB gene should be deleted or otherwise attenuated. One embodiment of the present invention is to delete or otherwise attenuate endogenous lipB gene. Another embodiment is to delete or otherwise attenuate endogenous lipB, lipA1, lipA2 genes of the present invention.

The invention provides various host strains that are dependent on light and exogenous lipoic acid for growth. With abundant lipoic acid, biomass formation will occur largely at the expense of ethanol production. When lipoic acid is depleted from the medium, although all other nutrients are in abundance, growth ceases as the rate of ethanol production sustainably increases. Additionally, it is desirable to have an inexpensive source of lipoic acid. Such source of lipoic acid may be provided from spent biomass.

### Fixed-Nitrogen Source (Ammonium and Nitrate) Directed Metabolic Switches

In one embodiment of the present invention, genetic regulatory elements for nitrogen metabolism are incorporated into metabolic switches. Cyanobacterial host cells use an endogenous system of the transcription regulator NtcA, nitrate reductase (Nar) and nitrite reductase (Nir) to convert nitrate to an ammonium ion (NH₄⁺). In the presence of urea, the host cell activates a metabolic pathway wherein endogenous urease protein is used to hydrolyze urea into ammonia and carbon dioxide. If only a nitrate is available, the nitrate will activate the nitrate/nitrite reductase pathway of host cells to generate pools of NH₄⁺. Therefore, metabolic pathways may be functionally linked to and activated when the nitrate/nitrite reductase metabolic pathway is active, yet the same pathway remains inactive when urea is presented exogenously.

One aspect of this invention is to employ as a control element nucleic acid promoter sequences regulating the expression of genes in a nitrate/nitrite reductase metabolic pathway. Such promoter sequences can be used to activate or up-regulate certain pathways generating ethanol or fatty acid derivative biosynthesis while inactivating or attenuating pathways used for the generation of biomass. In the presence of urea, pathways used for the generation of biomass will be active and other metabolic pathways specific for the generation of ethanol or other carbon-based products of interest will be inactive or attenuated. Alternatively, pathways used for the generation of biomass can be made active when in the presence of nitrate and other metabolic pathways to generate ethanol or other carbon-based products of interest will be inactive or attenuated

In one embodiment of the present invention, a strong promoter for the nir operon from Synechococcus sp. strain PCC 7942 (Pₙᵢᵣ) is used to control expression of a heterologous lipoamidase from Enterococcus faecalis (Accession #AAU94937). When presented with a nitrate source in the absence of urea or other preferentially utilized nitrogen sources, NtcA up-regulates Pₙᵢᵣ and Pₙₐᵣ-type promoters resulting in the expression of lipoamidase. Lipoamidase targets as a substrate lipoylated AceF, itself resulting from endogenous LipB-LipA1/LipA2 pathway and serving as an essential component of the pyruvate dehyodrogenase complex. Lipoamidase cleaves the lipoate group from AceF, inactivating Pdh. A host cell with an inactivated Pdh and a knock-out or attenuation of pyruvate:ferredoxin oxidoreductase and pyruvate formate lyase will be unable to synthesize acetyl-CoA from cellular pyruvate and will generate a pool of pyruvate. This cellular pool of pyruvate becomes available for diversion to the production of carbon based products of interest such as ethanol.

In another embodiment, a pdc gene is transcriptionally linked to a lipoamidase gene. Designing these genes to be transcriptionally linked ensures that sufficient levels of Pdc are available in the cell to initiate the Pdc-Adh ethanol production pathway when pyruvate levels accumulate from the expression of lipoamidase. Additional, potentially adverse effects of excess Pdc in the cell are avoided. Finally, the lack of Pdc protein in the cell when sufficient levels of urea are exogenously presented ensures that the cell will utilize carbon sources for biomass production.

Lipoamidase (#AAU94937) is potentially toxic to the host cell because of its high activity for lipoylated target substrates. Therefore, to increase or maintain overall production efficiency of the host cell construct, minimize cellular toxicity, and avoid the host cell tendency to mutate and inactivate the lpa gene, another embodiment of the present invention utilizes a less active lipoamidase truncated at amino acid residue 471. In a related embodiment, differential activity of lipoamidase is achieved by truncation of the full length protein at any one of amino acid positions 450 through amino acid 490 to match and optimize catalytic turnover of lipoamidase and endogenous lipoylation on target substrates specific to the engineered photautotrophic host cell.

In one embodiment, a host cell has lipoamidase expression controlled by a nitrate activated promoter. In a preferred embodiment, lipoamidase expression is controlled by heterologous Pₙᵢᵣ, being grown in media containing only urea until the desired cell density is achieved. In this method, biosynthetic pathways for converting pyruvate to pools of acetyl-CoA are active (allowing biomass to be efficiently generated) because the Pₙᵢᵣ promoter stays attenuated in the absence of nitrate. When the desired cell density is achieved, the growth media is exchanged so the nitrogen source for cellular use is a nitrate, for example sodium nitrate. Nitrate results in the expression of the Pₙᵢᵣ controlled lipoamidase gene (full length or truncated), by which expressed lipoamidase removes the lipoic acid moiety from AceF and deactivates the Pdh complex. The deactivated Pdh complex is unable to convert pyruvate to acetyl-CoA, enabling pyruvate to be preferentially metabolized as a dedicated substrate for expressed Pdc to generate pools of ethanol or any other desired carbon based product of interest. In a preferred embodiment, heterologous Adh activity is selected from Z. mobilis adhII, Z. mobilis adhII TS42, Z. mobilis adhB, and Moorella sp. HUC22-1 adhA, and heterologous Pdc activity is selected from Z. palmae and Z. mobilis.

In yet another embodiment, a host cell has endogenous and/or heterologous citrate synthase activity controlled by a nitrate activated promoter. In a preferred embodiment, citrate synthase expression is controlled by heterologous Pₙᵢᵣ, being grown in media containing only nitrate until the desired cell density is achieved. In this method, biosynthetic pathways for converting pools of acetyl-CoA to biomass production are active because the Pₙᵢᵣ promoter stays active in the presence of nitrate. When the desired cell density is achieved, the growth media is exchanged so the nitrogen source for cellular use is ammonia, for example from endogenous urease metabolism of exogenous urea. Upon exchange with urea, citrate synthase is no longer produced, disabling the citric acid cycle. The accumulating acetyl-CoA is preferentially metabolized to fatty acid derivatives that include carbon-based products of interest.

Alternatively, in another embodiment of the present invention, a mixed media containing fixed concentrations of both urea and a nitrate source is used. The urease metabolic pathway is preferentially activated over the nitrate conversion metabolic pathway. Host cells will use exogenous urea first until that nitrogen source is depleted, then activate the nitrate/nitrite reductase pathway to convert exogenous nitrate to NH₄⁺. Concomitant with activating Pₙᵢᵣ promoters, expression of lipoamidase (full length or truncated) and/or citrate synthase will be initiated. Variations in the proportion of urea and nitrate in the media are optimized to promote the desired biomass production before switching nitrogen sources and initiating alternative metabolic pathways for production of ethanol or other carbon based product of interest, including fatty acid derivatives. For example, ranges of urea could be from 5% to 99% of nitrogen sources, with nitrate being 95% to 1% of total fixed nitrogen. Preferably, a range of 15% to 30% urea is used, with a corresponding range of 70% to 85% nitrate, for the expression of lipoamidase. Preferably, the approximate range of 0.75-2.0 mM per desired OD₇₃₀ is used in the media (for example, if the desired final OD₇₃₀ is 10, the urea concentration upon inoculation of the media should be approximately 7.5-20 mM), with sufficient nitrate for the life of the host cells, for the expression of citrate synthase. The advantage to this method is that the cell biomass production is self-inactivated without the need for flushing and exchanging the growth media to switch to production of ethanol or desired carbon based product of interest.

### Selected or Engineered Microorganisms for the Production of Ethanol

Microorganisms include prokaryotic and eukaryotic microbial species from the domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. The engineered cell provided by the invention may be derived from eukaryotic plants, industrially important organisms including, but not limited to, Xanthomonas spp., Escherichia coli, Corynebacterium spp., Lactobacillus spp., Aspergillus spp., Streptomyces spp., Acetobacter spp., Penicillin spp., Bacillus spp., Pseudomonad spp., Clostridium spp., Zymomonas spp., Salmonella spp., Serratia spp., Erwinia spp., Klebsiella spp., Shigella spp., Enteroccoccus spp., Alcaligenes spp., Paenibacillus spp., Arthrobacter spp., Brevibacterium spp., algae, cyanobacteria, green-sulfur bacteria, green non-sulfur bacteria, purple sulfur bacteria, purple non-sulfur bacteria, extremophiles, yeast, fungi, engineered organisms thereof, and synthetic organisms. In certain related embodiments, the cell is light dependent or fixes carbon. In other related embodiments, the cell has autotrophic activity or photoautotrophic activity. In other embodiments, the cell is photoautotrophic in the presence of light and heterotrophic or mixotrophic in the absence of light. In other related embodiments, the engineered cell is a plant cell selected from the group consisting of Arabidopsis, Beta, Glycine, Jatropha, Miscanthus, Panicum, Phalaris, Populus, Saccharum, Salix, Simmondsia and Zea. In still other related embodiments, the engineered cell of the invention is an algae and/or cyanobacterial organism selected from the group consisting of Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spennatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thermosynechococcus, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

In yet other related embodiments, the engineered cell provided by the invention is derived from a Chloroflexus, Chloronema, Oscillochloris, Heliothrix, Herpetosiphon, Roseiflexus, and Thermomicrobium cell; a green sulfur bacteria selected from: Chlorobium, Clathrochloris, and Prosthecochloris; a purple sulfur bacteria is selected from: Allochromatium, Chromatium, Halochromatium, Isochromatium, Marichromatium, Rhodovulum, Thermochromatium, Thiocapsa, Thiorhodococcus, and Thiocystis; a purple non-sulfur bacteria is selected from: Phaeospirillum, Rhodobaca, Rhodobacter, Rhodomicrobium, Rhodopila, Rhodopseudomonas, Rhodothalassium, Rhodospirillum, Rodovibrio, and Roseospira; an aerobic chemolithotrophic bacteria selected from: nitrifying bacteria. Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria, Hydrogenobacter sp., iron and manganese-oxidizing and/or depositing bacteria, Siderococcus sp., and magnetotactic bacteria, Aquaspirillum sp; an archaeobacteria selected from: methanogenic archaeobacteria, Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic sulfur-Metabolizers such as Thermoproteus sp., Pyrodictium sp., Sulfolobus sp., Acidianus sp., Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

In other related embodiments, the engineered cell provided by the invention is derived from an extremophile that can withstand various environmental parameters such as temperature, radiation, pressure, gravity, vacuum, desiccation, salinity, pH, oxygen tension, and chemicals. These include hyperthermophiles, which grow at or above 80° C. such as Pyrolobus fumarii; thermophiles, which grow between 60-80° C. such as Synechococcus lividis; mesophiles, which grow between 15-60° C.; and psychrophiles, which grow at or below 15° C. such as Psychrobacter and some insects. Radiation tolerant organisms include Deinococcus radiodurans. Pressure tolerant organisms include piezophiles or barophiles which tolerate pressure of 130 MPa. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt tolerant organisms include halophiles (e.g., 2-5 M NaCl) Halobacteriacea and Dunaliella salina. pH tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas tolerant organisms, which tolerate pure CO₂, and metal tolerant organisms include metalotolerants such as Ferroplasma acidarmanus (e.g., Cu, As, Cd, Zn), Ralstonia sp. CH34 (e.g., Zn, Co, Cd, Hg, Pb) are also contemplated.

In yet other embodiments, the host cell is provided by the invention are derived from Arabidopsis thaliana, Panicum virgatum, Miscanthus giganteus, and Zea mays (plants), Botryococcus braunii, Chlamydomonas reinhardtii and Dunaliela salina (algae), Synechococcus sp. PCC 7002, Synechococcus sp. PCC 7942, Synechocystis sp. PCC 6803, and Thermosynechococcus elongatus BP-1 (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palusris (purple non-sulfur bacteria).

In still other embodiments, the engineered cell provided by the invention is a Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis cell.

In certain embodiments, the host cell provided by the invention is capable of conducting or regulating at least one metabolic pathway selected from the group consisting of photosynthesis, sulfate reduction, methanogenesis, acetogenesis, reductive TCA cycle, Calvin cycle, 3-HPA cycle and 3 HP/4 HB cycle.

A common theme in selecting or engineering a suitable organism is autotrophic fixation of carbon, such as CO₂ to products. This would cover photosynthesis and methanogenesis. Acetogenesis, encompassing the three types of CO₂ fixation; Calvin cycle, acetyl CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways (see, e.g., Fuchs, G. (1989) Alternative pathways of autotrophic CO₂ fixation, p. 365-382. In H. G. Schlegel, and B. Bowien (ed.), Autotrophic bacteria. Springer-Verlag, Berlin, Germany). The reductive pentose phosphate cycle (Calvin-Bassham-Benson cycle) represents the CO₂ fixation pathway in almost all aerobic autotrophic bacteria, for example, the cyanobacteria.

### Propagation of Selected Microoganisms

Methods for cultivation of photosynthetic organisms in liquid media and on agarose-containing plates are well known to those skilled in the art (see, e.g., websites associated with ATCC, and with the Institute Pasteur). For example, Synechococcus sp. PCC 7002 cells (available from the Pasteur Culture Collection of Cyanobacteria) are cultured in BG-11 medium (17.65 mM NaNO₃, 0.18 mM K₂HPO₄, 0.3 mM MgSO₄, 0.25 mM CaCl₂, 0.03 mM citric acid, 0.03 mM ferric ammonium citrate, 0.003 mM EDTA, 0.19 mM Na₂CO₃, 2.86 mg/L H₃BO₃, 1.81 mg/L MnCl₂, 0.222 mg/L ZnSO₄, 0.390 mg/L Na₂MoO₄, 0.079 mg/L CuSO₄, and 0.049 mg/L Co(NO₃)₂, pH 7.4) supplemented with 16 μg/L biotin, 20 mM MgSO₄, 8 mM KCl, and 300 mM NaCl (see, e.g., website associated with the Institute Pasteur, and Price G D, Woodger F J, Badger M R, Howitt S M, Tucker L. “Identification of a SulP-type bicarbonate transporter in marine cyanobacteria. Proc Natl. Acad. Sci. USA (2004) 101(52):18228-33). Typically, cultures are maintained at 28° C. and bubbled continuously with 5% CO2 under a light intensity of 120 μmol photons/m2/s. Alternatively, Synechococcus sp. PCC 7002 cells are cultured in A⁺ medium as previously described [Frigaard N U et al. (2004) “Gene inactivation in the cyanobacterium Synechococcus sp. PCC 7002 and the green sulfur bacterium Chlorobium tepidum using in vitro-made DNA constructs and natural transformation,” Methods Mol. Biol., 274:325-340].

Thermosynechococcus elongatus BP-1 (available from the Kazusa DNAResearch Institute, Japan) is propagated in BG11 medium supplemented with 20 mM TES-KOH (pH 8.2) as previously described [Iwai M, Katoh H, Katayama M, Ikeuchi M. “Improved genetic transformation of the thermophilic cyanobacterium, Thermosynechococcus elongatus BP-1.” Plant Cell Physiol (2004). 45(2):171-175)]. Typically, cultures are maintained at 50° C. and bubbled continuously with 5% CO₂ under a light intensity of 38 μmol photons m⁻² s⁻¹. T. elongatus BP-1 can be grown in A⁺ medium also.

Chlamydomonas reinhardtii (available from the Chlamydomonas Center culture collection maintained by Duke University, Durham, N.C.,) are grown in minimal salt medium consisting of 143 mg/L K₂HPO₄, 73 mg/L KH₂PO₄, 400 mg/L NH₄NO₃, 100 mg/L MgSO₄-7H₂O, 50 mg/L CaCl₂-2H₂0, 1 mL/L trace elements stock, and 10 mL/L 2.0 M MOPS titrated with Tris base to pH 7.6 as described (Geraghty A M, Anderson J C, Spalding M H. “A 36 kilodalton limiting-CO₂ induced polypeptide of Chlamydomonas is distinct from the 37 kilodalton periplasmic anhydrase.” Plant Physiol (1990). 93:116-121). Typically, cultures are maintained at 24° C. and bubbled with 5% CO₂ in air, under a light intensity of 60 μmol photons m⁻² s⁻¹.

The above define typical propagation conditions. As appropriate, incubations are performed using alternate media or gas compositions, alternate temperatures (5-75° C.), and/or light fluxes (0-5500 μmol photons m⁻² s⁻¹).

Light is delivered through a variety of mechanisms, including natural illumination (sunlight), standard incandescent, fluorescent, or halogen bulbs, or via propagation in specially-designed illuminated growth chambers (for example Model LI15 Illuminated Growth Chamber (Sheldon Manufacturing, Inc. Cornelius, Oreg.). For experiments requiring specific wavelengths and/or intensities, light is distributed via light emitting diodes (LEDs), in which wavelength spectra and intensity can be carefully controlled (Philips).

Carbon dioxide is supplied via inclusion of solid media supplements (i.e., sodium bicarbonate) or as a gas via its distribution into the growth incubator or media. Most experiments are performed using concentrated carbon dioxide gas, at concentrations between 1 and 30%, which is directly bubbled into the growth media at velocities sufficient to provide mixing for the organisms. When concentrated carbon dioxide gas is utilized, the gas originates in pure form from commercially-available cylinders, or preferentially from concentrated sources including off-gas or flue gas from coal plants, refineries, cement production facilities, natural gas facilities, breweries, and the like.

### Transformation of Selected Microorganisms

Synechococcus sp. PCC 7002 cells are transformed according to the optimized protocol previously described [Essich E S, Stevens Jr., E, Porter R D “Chromosomal Transformation in the Cyanobacterium Agmenellum quadruplicatum”. J Bacteriol (1990). 172(4):1916-1922]. Cells are grown in Medium A (18 g/L NaCl, 5 g/L MgSO₄. 7H₂O, 30 mg/L Na₂EDTA, 600 mg/L KCl, 370 mg/L CaCl₂.2 H₂O, 1 g/L NaNO₃, 50 mg/L KH₂PO₄, 1 g/L Trizma base pH 8.2, 4 μg/L Vitamin B₁₂, 3.89 mg/L FeCl₃. 6 H₂O, 34.3 mg/L H₃BO₃, 4.3 mg/L MnCl₂.4 H20, 315 μg/L ZnCl₂, 30 μg/L MoO₃, 3 μg/L CuSO₄.5 H₂0, 12.2 μg/L CoCl₂.6 H₂0) [Stevens S E, Patterson COP, and Myers J. “The production of hydrogen peroxide by green algae: a survey.” J. Phycology (1973). 9:427-430] plus 5 g/L of NaNO₃ to approximately 108 cells/mL. Nine volumes of cells are mixed with 1 volume of 1-10 μg/mL DNA in 0.15 M NaCl/0.015 M Na₃citrate and incubated at 27-30° C. for 3 hours before addition of 1 volume of DNaseI to a final concentration of 10 μg/mL. The cells are plated in 2.5 mL of 0.6% medium A overlay agar that was tempered at 45° C. and incubated. Cells are challenged with antibiotic by under-laying 2.0 mL of 0.6% medium A agar containing appropriate concentration of antibiotic with a sterile Pasteur pipette. Transformants are picked 3-4 days later. Selections are typically performed using 200 μg/ml kanamycin, 8 μg/ml chloramphenicol, 10 μg/ml spectinomycin on solid media, whereas 150 μg/mlkanamycin, 7 μg/ml chloramphenicol, and 5 μg/ml spectinomycin are employed in liquid media.

T. elongatus BP-1 cells are transformed according to the optimized protocol previously described (vide supra).

E. coli are transformed using standard techniques known to those skilled in the art, including heat shock of chemically competent cells and electroporation (Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, N.Y.; and Current Protocols in Molecular Biology, F. M. Ausubel et al., eds., Current Protocols, a joint venture between Greene Publishing Associates, Inc. and John Wiley & Sons, Inc., (through and including the 1997 Supplement)).

The biosynthetic pathways as described herein are first tested and optimized using episomal plasmids described above. Non-limiting optimizations include promoter swapping and tuning, ribosome binding site manipulation, alteration of gene order (e.g., gene ABC versus BAC, CBA, CAB, BCA), co-expression of molecular chaperones, random or targeted mutagenesis of gene sequences to increase or decrease activity, folding, or allosteric regulation, expression of gene sequences from alternate species, codon manipulation, addition or removal of intracellular targeting sequences such as signal sequences, and the like.

Each gene is optimized individually, or alternately, in parallel. Functional promoter and gene sequences are subsequently integrated into the E. coli chromosome to enable stable propagation in the absence of selective pressure (i.e., inclusion of antibiotics) using standard techniques known to those skilled in the art.

### Ethanol and Fatty Acid Derivative Production

In embodiments applicable to auxotrophic host cells described herein, the general method for ethanol/fatty acid derivative biosynthesis comprises culturing the engineered host cell to be auxotrophic for a specific exogenous compound, for example nitrate/urea or lipoic acid, and culturing said host to be depleted of the specific exogenous compound whereby a desired end-product (e.g., ethanol and fatty acid derivatives including alkanes) is produced. In an alternative embodiment, a general method for desired end-product biosynthesis comprises engineering a host cell to be auxotrophic for a specific exogenous compound, for example nitrate/urea or lipoic acid, culturing the host cell in a growth medium with the specific exogenous compound to increase biomass and attenuate desired end-product biosynthesis, and then culturing the host cell in a growth medium without the same specific exogenous compound to attenuate biomass growth and synthesize desired end-products.

A host cell's maximal potential productivity for carbon-based products can be estimated for photoautotrophs by determining biomass productivity of a wild type strain from which the host cell of the invention is derived. In a closed, controlled system, photoautotophs will experience a small and brief period of exponential cell population growth before entering a period of linear cell population growth (decreased photon penetration due to cell population densities limit the population growth, and a linear population increase is demonstrated before reaching a plateaued, stationary growth phase). In measuring the slope of a line best fitted to the linear portion of a population growth curve, the rate of increase in biomass can be evaluated as follows: Subjecting a known mass of desiccated cells to calorimetric techniques will determine how much energy (in Joules) is in the mass of cells. Therefore, maximal productivity (in terms of biomass) can be calculated by multiplying the growth rate by energy content of the cells. For example, assuming a linear growth rate of 30 mg/liter/hour and a calorimetric content of 21,000 Joules/gram, the biomass productivity is (0.03 gram/liter/hour)×((21,000 Joules/gram)=630 Joules/liter/hour. Assuming a calorimetric content of ethanol to be 30,000 Joules/gram (available from published research literature), and complete redirection of cellular resources from biomass production to production of carbon-based products, maximal potential productivity is (630 Joules/liter/hour)/(30,000 Joules/gram)=21 mg/liter/hour.

Other alcohols (short chain, long chain, branched or unsaturated) and fatty acid derivatives can be produced by identifying the relevant pathways, providing the organism with certain growth-essential nutrients and using the metabolic switch as described herein. See for example U.S. Pat. No. 7,794,969 which is incorporated herein in its entirety. Alcohols can be used as fuels directly or they can be used to create an ester, i.e. the A side of an ester as described above. Such ester alone or in combination with the other fatty acid derivatives described herein are useful a fuels.

Standard culture buffers and transformed phototrophic host cells containing relevant engineered components are incubated at applicable temperatures and CO₂ flux in a Multitron II (Infors) shaking photoincubator. For the phototrophic host strains of the present invention, cells are incubated under continuos light conditions (˜100 μM photons m⁻² s⁻¹) for 20 hours at 37° C., 150 rpm in 2.0% CO₂-enriched air.

### Productivity Evaluation

Generally, the products of interest produced from a batch culture or photobioreactor can be analyzed by any of the standard analytical methods, such as gas chromatography (GC), mass spectrometry (MS) gas chromatography-mass spectrometry (GCMS), and liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G., R. O. Dunn, and M. O. Bagby. 1997. Biodiesel: The use of vegetable oils and their derivatives as alternative diesel fuels. Am. Chem. Soc. Symp. Series 666: 172-208), titration for determining free fatty acids (Komers, K., F. Skopal, and R. Stloukal. 1997. Determination of the neutralization number for biodiesel fuel production. Fett/Lipid 99(2): 52-54), enzymatic methods (Bailer, J., and K. de Hueber. 1991. Determination of saponifiable glycerol in “bio-diesel.” Fresenius J. Anal. Chem. 340(3): 186). Other physical property-based methods, wet chemical methods, etc. well known to those in the art can be used to analyze the levels and the identity of the product produced by the organisms used in a photobioreactor.

To calculate the productivity the following assumptions are made: Radiation: photosynthetically active radiation (PAR) fraction of total solar radiation ˜47%, historical average PAR at ground based on NREL 1991-2005 datasets, assumes future radiation characteristics will be consistent with historic values; Production: production rate is linear with radiation intensity, well-documented photon utilization is 8 photons/CO₂ fixed into biomass (Pirt, S J 1983, Biotechnol Bioeng, 25: 1915-1922), 85% of PAR striking a photobioreactor system enters the culture, 85% of PAR photons entering the photobioreactor are available for conversion, 15% lost to photoinhibition & radiation when culture not at operating temperature, Estimate 3 days of culture growth followed by 8 weeks of production; 95% online production, Estimate 5% of photo synthetic energy dedicated to cell maintenance (Pirt S J 1965 Proc Roy Soc 163: 224-231). Method of calculating ethanol productivity based on ethanol concentration in the culture and the stripping rate: The ethanol concentration in a photobioreactor culture is a function of two quantities: (a) The production rate (kₚ), which is the rate of increase of ethanol concentration in the liquid with time:

d[Ethanol]/dt=kₚ Eqn. 2

and (b) the stripping rate (s), due to the volatility of ethanol, and will be continuously leaving the liquid in the form of vapor. The rate at which the ethanol leaves a photobioreactor or batch culture is a function of the concentration of ethanol in the liquid and a variety of other factors such as temperature, airflow, etc. For our purposes, all other factors are held fixed hence we can think of the rate of ethanol loss being solely dependent on the liquid concentration, i.e:

d[Ethanol]/dt=−s[Ethanol] Eqn. 3

Combining the two equations, we can write:

d[Ethanol]/dt=kₚ−s[Ethanol] Eqn 4

Note that in the above equation, the production rate kₚ is time independent which is clearly false. In reality, it would depend on time via the density of the culture and the light regime. However, as long as we treat the production rate kₚ as an average production rate between measurements, the relation is valid.

The equation is a basic first order equation and can be easily solved to obtain:

kₚ=s[Ethanol(t)]e^{ts}−s[Ethanol(t=0)]/(e^{ts}−1) Eqn. 5

This gives a production rate that is in terms of concentration of ethanol per unit time for the incident light intensity at which the experiment was conducted. This has to be multiplied by the reactor volume to obtain the production rate in terms of grams of ethanol per unit time. Units can then be converted to suitable time units such as day instead of hour. For example, in our case, we define the stripping rate in units of hour⁻¹ and our reactor of volume V covers and area of 0.5 meter². Therefore our production rate (in grams per square meter per day) is given by:

Production rate=2kₚV*24 Eqn. 6

where the production rate is in units of grams/meter²/day at the incident light intensity at which the experiment was conducted.

### Processing & Separation

Ethanol can be easily separated from the culture solution and distilled by those of skill in the art, according to any known method. Fractional distillation can concentrate ethanol to approximately 95.6% by volume (89.5 mole %). Absolute alcohol can be obtained by adding a small quantity of benzene and then subjecting the ethanol to further fractional distillation.

Absolute alcohol can also be produced by desiccation using glycerol, or adsorbents such as starch or zeolites, which adsorb water preferentially. Azeotropic distillation and extractive distillation techniques may also be used.

### EXAMPLES

The examples below are provided herein for illustrative purposes and are not intended to be restrictive.

### Example 1

### Metabolic Switch Using Lipoic Acid Controlled Pdh in JCC1581

The ethanologen used in this study is the strain JCC138::PAQ7_P(cI)_adhAm_kan::PAQ3_P(nir07)_pdcZm_adhA_spec which is referred to as JCC1581. In this strain both lipA1 and lipA2 genes were knocked out, therefore disabling the endogenous lipoylation pathway of PDH. In order to allow the strain to grow, E. coli lplA was expressed from the P(cI) promoter. In addition, aas1 (acyl-ACP synthetase) was also knocked out to avoid the potential channeling of exogenous lipoic acid into the acyl-ACP pool, which would be toxic to the cell. The final lipoic acid auxotroph strain is JCC1581::aas1_P(cI)_lplA_{—}E. coli_gent ΔlipA1_ery ΔlipA2_zeo. The strain was tested for segregation by colony PCR. Results showed the presence of the lplA, erythromycin and zeomycin at the aas1, lipA1 and lipA2 loci respectively.

The JCC138, JCC1581, and JCC1581 lipoic acid auxotroph strains (Table 1) were inoculated from single colonies into 15 ml test tubes and grown in 7 ml of low-salt A+, 3 mM urea (Table 3) and 0.1 μg/mllipoic acid with the appropriate antibiotic (Table 1). Test tubes were grown for 4 days with constant shaking. All flasks contain the same volume of either lipoic acid/DMSO or DMSO alone. On the fourth day, optical densities at 730 nm were measured for each seed culture and the appropriate volume for each strain was used to inoculate 30 ml of medium to an OD730=0.05. The indicated volumes were removed from each test tube culture, centrifuged and washed twice in 1 ml of low-salt JB2.1 media (Table 4) to remove traces of lipoic acid. Each seed culture was then inoculated into 30 ml of JB2.1 media supplemented with either lipoic acid diluted in DMSO or DMSO alone in 125 ml flasks (Table 2).

TABLE 1
Starter cultures

| | | | | | Final lipoic |
|---|---|---|---|---|---|
| | | | Media | | acid/DMSO |
| Flask | | | volume | Antibiotic concentration (μG/mL, | conc. |
| # | Construct | Media | (mL) | denoted in superscript) | (mg/mL) |
| 1 | JCC138 | Low salt | 7 | none | 0.0001 |
| | | A+/3 mM | | | |
| | | urea | | | |
| 2 | JCC1581 | Low salt | 7 | spectinomycin¹⁰⁰/kanamycin⁵⁰ | 0.0001 |
| | | A+/3 mM | | | |
| | | urea | | | |
| 3 | JCC1581_auxotroph | Low salt | 7 | spectinomycin¹⁰⁰/kanamycin⁵⁰/ | 0.0001 |
| | | A+/3 mM | | gentamycin²⁵/ | |
| | | urea | | erythromycin²⁰/zeomycin⁵⁰ | |

TABLE 2
Growth cultures

| | | | | | Final lipoic |
|---|---|---|---|---|---|
| | | | Media | | acid/DMSO |
| Flask | | | volume | Antibiotic concentration (μG/mL, | conc. |
| # | Construct | Media | (mL) | denoted in superscript) | (mg/mL) |
| 1 | JCC138 | Low salt | 30 | none | 0.0000 |
| | | JB2.1+/3 mM | | | |
| | | urea | | | |
| 2 | JCC138 | Low salt | 30 | none | 0.0002 |
| | | JB2.1+/3 mM | | | |
| | | urea | | | |
| 3 | JCC1581 | Low salt | 30 | spectinomycin¹⁰⁰/kanamycin⁵⁰ | 0.0000 |
| | | JB2.1+/3 mM | | | |
| | | urea | | | |
| 4 | JCC1581 | Low salt | 30 | spectinomycin¹⁰⁰/kanamycin⁵⁰ | 0.0002 |
| | | JB2.1+/3 mM | | | |
| | | urea | | | |
| 5 | JCC1581_auxotroph | Low salt | 30 | spectinomycin¹⁰⁰/kanamycin⁵⁰/ | 0.0000 |
| | | JB2.1+/3 mM | | gentamycin²⁵/ | |
| | | urea | | erythromycin²⁰/zeomycin⁵⁰ | |
| 6 | JCC1581_auxotroph | Low salt | 30 | spectinomycin¹⁰⁰/kanamycin⁵⁰/ | 0.0002 |
| | | JB2.1+/3 mM | | gentamycin²⁵/ | |
| | | urea | | erythromycin²⁰/zeomycin⁵⁰ | |

TABLE 3
Low salt A+ media

| Low salt A+ media: | (g/L, final) |
|---|---|
| Sodium chloride | 5.000 |
| Potassium chloride | 0.6 |
| Sodium nitrate | 1.0 |
| Magnesium sulfate heptahydrate | 5.0 |
| Potassium phosphate monobasic | 0.05 |
| EDTA, disodium salt dehydrate | 0.029 |
| Iron (III) chloride hexahydrate | 0.004 |
| Tris/THAM ® | 1.00 |
| Calcium chloride, anhydrous | 0.266 |
| Boric acid | 0.343 |
| Manganese chloride tetrahydrate | 0.00432 |
| Zinc chloride | 0.000315 |
| Molybdenum (VI) oxide | 0.00003 |
| Copper (II) sulfate pentahydrate | 0.000003 |
| Cobalt (II) chloride hexahydrate | 0.00001215 |

TABLE 4
Low salt JB2.1 media

| Low salt JB2.1 media | (g/L, final) |
|---|---|
| Sodium chloride | 5.00 |
| Potassium chloride | 0.60 |
| Sodium nitrate | 3.5 |
| Magnesium sulfate heptahydrate | 5.00 |
| Potassium phosphate monobasic | 0.20 |
| EDTA, disodium salt dehydrate | 0.029 |
| Iron (III) citrate hydrate | 0.014 |
| Tris/THAM ® | 1.00 |
| Urea | 0.18 |
| Calcium chloride, anhydrous | 0.266 |
| Boric acid | 0.034 |
| Manganese chloride tetrahydrate | 0.0043 |
| Zinc chloride | 0.00032 |
| Molybdenum (VI) oxide | 0.00003 |
| Copper (II) sulfate pentahydrate | 0.000003 |
| Cobalt (II) chloride hexahydrate | 0.000012 |

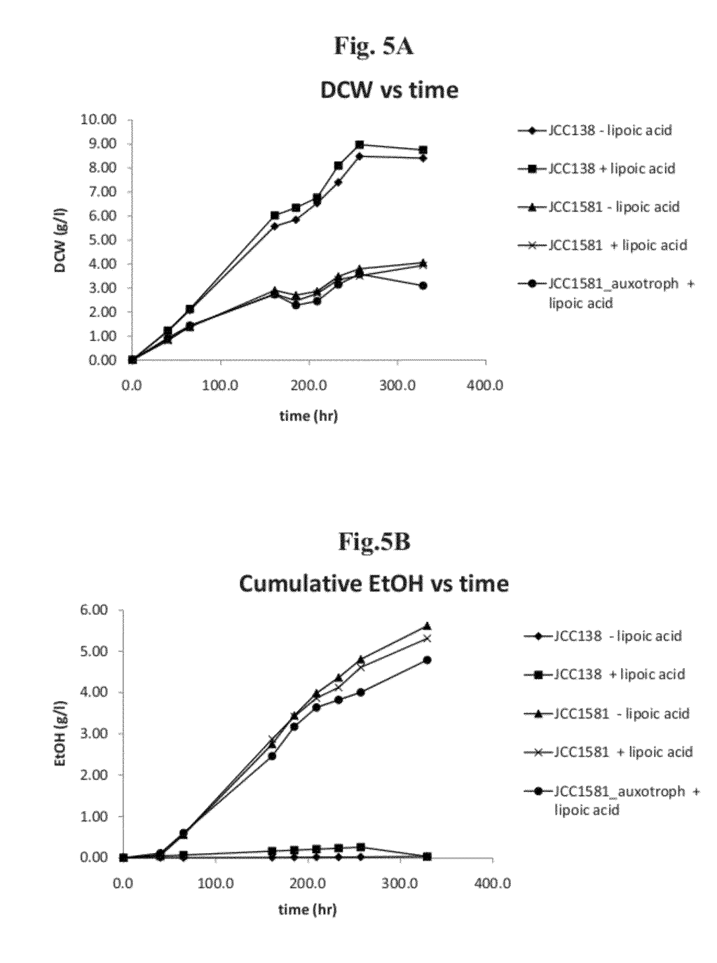

The strain JCC1581::aas1_P(cI)_lplA_{—}E. coli_gent ΔlipA1_ery ΔlipA2_zeo was tested for lipoic acid auxotrophy and shown to be unable to grow in the absence of exogenous lipoic acid (FIG. 5A, Table 5). The strain was also shown to have similar growth curve and ethanol productivity as JCC1581 in the presence of lipoic acid (FIG. 5A,B, Tables 5 and 6). It was also shown that only a very low amount of lipoic acid is required for the cells to grow. After washing the seed cultures and removing the lipoic acid from the A+media, the cells still grow for at least 24 hours in lipoic acid depleted JB2.1 media before starting to bleach, which is probably due to its uptake inside the cell.

TABLE 5

| | Dry Cell Weight (g/l) | | | | |
|---|---|---|---|---|---|
| | JCC138_A | JCC138_A | JCC1581_B | JCC138_B | |
| Time | (−) lipoic | (+) lipoic | (−) lipoic | (+) lipoic | JCC1581_auxotroph_B |
| (hr) | acid | acid | acid | acid | (+) lipoic acid |
| 0.0 | 0.02 | 0.02 | 0.02 | 0.02 | 0.02 |
| 40.0 | 1.20 | 1.23 | 0.87 | 0.81 | 0.92 |
| 65.0 | 2.08 | 2.13 | 1.38 | 1.42 | 1.44 |
| 161.0 | 5.57 | 6.03 | 2.90 | 2.75 | 2.74 |
| 185.0 | 5.85 | 6.35 | 2.71 | 2.47 | 2.29 |
| 209.0 | 6.54 | 6.77 | 2.87 | 2.77 | 2.46 |
| 233.0 | 7.41 | 8.10 | 3.48 | 3.35 | 3.15 |
| 257.0 | 8.49 | 8.97 | 3.80 | 3.51 | 3.59 |
| 329.0 | 8.41 | 8.75 | 4.06 | 3.95 | 3.10 |

TABLE 6

| | Cumulative EtOH (g/l) | | | | |
|---|---|---|---|---|---|
| | JCC138_A | JCC138_A | JCC1581_B | JCC138_B | |
| Time | (−) lipoic | (+) lipoic | (−) lipoic | (+) lipoic | JCC1581_auxotroph_B |
| (hr) | acid | acid | acid | acid | (+) lipoic acid |
| 0.0 | 0.00 | 0.00 | 0.00 | 0.00 | 0.00 |
| 40.0 | 0.00 | 0.00 | 0.07 | 0.06 | 0.11 |
| 65.0 | 0.00 | 0.00 | 0.55 | 0.57 | 0.60 |
| 161.0 | 0.01 | 0.00 | 2.75 | 2.88 | 2.46 |
| 185.0 | 0.01 | 0.00 | 3.45 | 3.42 | 3.17 |
| 209.0 | 0.02 | 0.00 | 3.98 | 3.87 | 3.64 |
| 233.0 | 0.02 | 0.00 | 4.36 | 4.12 | 3.82 |
| 257.0 | 0.02 | 0.00 | 4.81 | 4.61 | 4.01 |
| 329.0 | 0.03 | 0.03 | 5.62 | 5.31 | 4.79 |

### Example 2

### Metabolic Switch Using Lipoic Acid Controlled Pdh

The following strain is constructed by standard homologous recombination techniques. Wild-type Synechococcus sp. PCC 7002, the starting material, is obtained from the Pasteur Collection or ATCC. Gene deletion constructs made synthetically may be obtained from DNA 2.0 or by PCR, and oligonucleotides for PCR and sequence confirmation from IDT. Lipoic acid is obtained from Sigma.

Synechococcus 7002 is grown for 48 h from colonies in an incubated shaker flask at 30° C. at 1% CO₂ to an OD₇₃₀ of 1 in A⁺ medium described in Frigaard, et al., (Methods Mol Biol 274:325-340 (2004)). 500 μL of culture is added to a test-tube with 30 μL of 1-5 μg of DNA prepped from a Qiagen Qiaprep Spin Miniprep Kit (Valencia, Calif.) for each construct. Cells are incubated bubbling in 1% CO₂ at approximately 1 bubble every 2 seconds for 4 hours. 200 μL of cells are plated on A⁺ medium plates with 1.5% agarose and grown at 30° C. for two days in low light. 10 μg/mL of spectinomycin is underplayed on the plates. Resistant colonies are visible in 7-10 days.

TABLE 7

| Gene to be Deleted | Function |
|---|---|
| A0785(lipA1)::pdc-adh | Abolishes endogenous lipoyl synthase |
| | activity → lipoic acid auxotroph; |
| | EtOH+ |
| A1577(lipA2) | Abolishes endogenous lipoyl synthase |
| | activity → lipoic acid auxotroph |
| A1443(nifJ) | Abolishes alternative route from |
| | pyruvate to ACoA → PDH-dependent |
| | viability |
| G0164(ldhA) | Abolishes alternative potential |
| | route for pyruvate |
| A0250(pps) | Abolishes alternative potential |
| | route for pyruvate |

*Underlined genes are heterologous

Table 7, above, shows the specific genes to be deleted. The lipA deletions result in lipoic acid auxotrophy, the nifJ deletion in complete dependence on Pdh for converting pyruvate to acetyl-CoA, the ldhA deletion in elimination of lactate dehyrogenase activity (lactate⇄pyruvate), and the pps deletion in elimination of phosphoenolpyruvate synthase activity. These genes and reactions are discussed in the literature by, e.g., Yokota et al., (1994), App. Micro. Biotech., 41:638-646; and Li et al. (2006), J. Biol. Chem., 122:254-266.

### Example 3

### Metabolic Switch Using Pₙᵢᵣ Controlled Lipoamidase

The following strain is constructed by standard homologous recombination techniques. Wild-type Synechococcus sp. PCC 7002, the starting material, is obtained from the Pasteur Collection or ATCC. Gene deletion constructs made synthetically may be obtained from DNA2.0 or by PCR, and oligonucleotides for PCR and sequence confirmation from IDT. Synechococcus 7002 is grown for 48 h from colonies in an incubated shaker flask at 30° C. at 1% CO₂ to an OD₇₃₀ of 1 in A⁺ medium described in Frigaard, et al. (Methods Mol Biol 274:325-340 (2004)). 500 μL of culture is added to a test-tube with 30 μL of 1-5 μg of DNA prepped from a Qiagen Qiaprep Spin Miniprep Kit (Valencia, Calif.) for each construct. Cells are incubated bubbling in 1% CO₂ at approximately 1 bubble every 2 seconds for 4 hours. 200 μL of cells are plated on A⁺ medium plates with 1.5% agarose and grown at 30° C. for two days in low light. 10 μg/mL of spectinomycin is underlayed on the plates. Resistant colonies are visible in 7-10 days.

TABLE 8

| Gene to be Overexpressed | Function |
|---|---|
| Enterococcus faecalis Lipoamidase | Removes essential lipoyl group |
| (Ef Lpa) gene code AY735444 | from pyruvate dehydrogenase (PDH) |
| | complex. PDH inactivation reduces |
| | flux of pyruvate to acetyl-CoA. |

*Underlined genes are heterologous

Table 8, above, shows the specific gene to be heterologously expressed. Expression of Ef Lpa will reduce activity of the pyruvate dehydrogenase complex by cleaving the essential lipoyl prosthetic group from the E2 subunit (encoded by the aceF gene, SYNPCC7002_A0110). Consequently, the flux of pyruvate to acetyl-CoA is much reduced in all systems tested. These genes and reactions are discussed in the literature by, e.g., Spalding, M. D. and Prigge, S. T. (2009) PLoS One 4:e7392, and Jiang, Y. and Cronan, J. E. (2005) J Biol Chem. 280:2244-56.

### Example 4

### Metabolic Switch Using Pₙᵢᵣ Controlled Citrate Synthase

The following strain is constructed by standard homologous recombination techniques. The starting materials are wild-type Synechococcus sp. PCC 7002 (JCC138) obtained from the Pastuer Collection or American Type Culture Collection. Promoter replacement constructs made synthetically may be obtained from DNA2.0 or by PCR, oligonucleotides for PCR and sequence confirmation from IDT. Urea is obtained from Sigma.

Promoter replacement constructs were naturally transformed into JCC138 using standard cyanobacterial transform protocols familiar to those having ordinary skill in the art. Briefly, 5-10 μg of plamid DNA was added to 1 mL of neat JCC138 culture that had been grown to an OD₇₄₀ of approximately 1.0. The cell/DNA mixture was incubated at 37° C. for four hours in the dark with gentle mixing, plated on to A+ plates and incubated in a photo-incubater (Percival) for 24 hours. Thereafter, gentamycin to a final concentration of 25 μg/mL was underlaid on the plates. Gentamycin resistant colonies appeared after 5-8 days of further incubation under 24 hour light conditions (˜100 μmol photons m⁻² s⁻¹. Following one round of colony purification on A+ plates supplemented with 25 μg/mL gentamycin, single colonies of each of the six transformed strains were grown in test tubes for 4-8 days at 37° C., 150 rpm in 3% CO₂ enriched are at ˜100 μmol photons m₋₂ s₋₁ in a Multitron II (Infors) shaking photo-incubator. The growth medium used for liquid culture was A+ with 25 μg/mL gentamycin.

TABLE 9

| Promoter swap | Function |
|---|---|
| A2623 (citrate synthase, gltA) | Puts citrate synthase gene (A2623) |
| endogenous promoter replaced | under control of P(nirA) which allows |
| by P(nirA) from PCC7942 | nitrate induction and urea or ammonia |
| (Synpcc7942_1240) | repression. |

Table 9 above shows the specific promoter replacement. The endogenous gltA promoter is replaced by the promoter from Synechococcus PCC7942 nirA (P(nirA)). This allows the gltA gene to be induced in nitrate-containing media and repressed in media containing urea or ammonia. As flux into the TCA cycle via citrate synthase is a major pathway utilizing acetyl-CoA, repression of citrate synthase maximizes the pool of acetyl-CoA available for alternate biosynthesis pathways, including fatty acid derivatives. These reactions and others are mapped in FIG. 4.

As the TCA cycle also provides precursors for many other biosynthetic pathways including several amino acids, repression of citrate synthase (the major point at which new carbon enters the cycle) slows or prevents cell growth and biomass accumulation. Therefore, cells are grown in nitrate containing media to an optical density at 730 nm (OD₇₃₀) of ˜3. Addition of urea to the media at this point prevents further TCA cycle activity, slowing growth and DCW (“dry cell weight;” used as a measure of biomass production) accumulation. This allows increased fatty acid biosynthesis from the larger pool of acetyl-CoA which results.

A 221 bp fragment of sequence 5′ to the nirA start codon as described is used (S. Maeda et al. (1998). Cis-Acting Sequences Required for NtcB-Dependent, Nitrite-Responsive Positive Regulation of the Nitrate Assimilation Operon in the Cyanobacterium Synechococcus sp. Strain PCC 7942. J Bacteriol. 180: 4080-4088).

When grown in the presence of 0.01-0.2% n-butanol, strain JCC803 produces fatty acid butyl esters (FABEs). We examined FABE production by JCC803 with a derivative of JCC803 in which the endogenous citrate synthase (gltA) promoter is replaced with P(nirA) (P(nirA)-gltA). We extracted FABEs from cell pellets after 336 hr after inoculation and found that JCC803 produced FABEs at 365 mg/L and P(nirA)-gltA strain produced FABEs at 471 mg/L, an increase of 29%. Production of 1-nonadecene also increased in P(nirA)-gltA strain to 15.3 mg/L compared to 12.1 mg/L in the JCC803 parent strain. Interestingly, DCW accumulation in the P(nirA)-gltA strain was not strongly impacted by repression of citrate synthase suggesting that we can increase the available pool of acetyl-CoA for fatty acid biosynthesis without strongly impacting other biosynthetic pathways.

### Example 5

### Metabolic Switch Using Lipoic Acid Controlled Pdh

The following strain is constructed by standard homologous recombination techniques as set forth in, for example, Sambrook et al., (1989) Molecular Cloning: A Laboratory Manual and using common laboratory techniques well known to those in the art. Additionally, Eikmanns, et al., (1994) Microbiology Vol. 140:1817-1828 and Blombach et al., (2007) Applied and Environmental Microbiology Vol. 73: No. 7:2079-2084 set forth techniques and laboratory protocols particular relevant to the transformation and culture of the host cell described. Stock strain Escherichia coli K-12 is obtained from the Pasteur Collection or ATCC. Gene deletion constructs are made synthetically for the lipA operon (lipA1) while preserving lipA2, encoding for the dihydrolipoyltransacetylase component of pyruvate dehydrogenase, and thus allowing for the pdh enzyme to become lipoylated with exogenously supplied lipoic acid. Additional deletions to render the host cell auxotrophic for lipoic acid are for the ydbK, pps and ldhA loci. Finally, the acyl-ACP synthetase (aas) deletion prevents potential toxic build-up of lipoyl ACP in cell membranes. The synthetic deletion constructs may be obtained from DNA2.0 or by PCR, and oligonucleotides for PCR and sequence confirmation from IDT. Lipoic acid is obtained from Sigma.

TABLE 10

| Gene to be Deleted | Function |
|---|---|
| b2836(aas) | Abolishes acyl ACP synthetase activity |
| b1378(ydbK) | Abolishes alternate route for pyruvate metabolism |
| | pyruvate:flavodoxin oxidoreductase activity |
| b0630(lipA1) | Lipoic acid auxotrophy |
| b1702(pps) | Abolishes alternative route for pyruvate metabolism |
| b1380(ldhA) | Abolishes alternative route for pyruvate metabolism |

*Underlined genes are heterologous

Table 10, above, shows the specific genes to be deleted. The lipA1 deletions result in lipoic acid auxotrophy, the ydbK deletion in complete dependence on Pdh for converting pyruvate to acetyl-CoA, the ldhA deletion in elimination of lactate dehyrogenase activity (lactate→pyruvate), and the pps deletion in elimination of phosphoenolpyruvate synthase activity. Homologues to these genes and reactions are discussed in the literature by, e.g., Yokota et al., (1994), App. Micro. Biotech., 41:638-646; and Li et al. (2006), J. Biol. Chem., 122:254-266.

Plasmids are constructed to express heterologous AdhA and Pdc from Z. palmae or Z. mobilis and transformed into host cells according to the references contained herein.

All publications and patent documents cited herein are hereby incorporated by reference in their entirety for all purposes to the same extent as if each were so individually denoted.

### Example 6

### Evaluation of Productivity Changes by a Metabolic Switch in a Cultured Host Cell

Metabolic switches are designed to be applied during the linear phase of growth of a host strain that has been metabolically engineered to produce one or more carbon-based products of interest. Without a metabolic switch, during the linear growth phase an engineered host cell exhibits production of biomass (i.e., production of dry cell weight, DCW) and production of engineered carbon-based products. Described herein is the effect of a metabolic switch on the productivity of products of interest attained following full induction of an engineered heterologous pathway during the linear growth phase.

Following entry of the host strain into the linear growth phase, but prior to the triggering of the metabolic switch, i.e., prior to the change in concentration of an exogenous compound in the medium that controls the metabolic switch, the host generates DCW with productivity m (grams DCW/liter/hour, “gm_{dcw}/l/hr”) and products of interest with productivity p (grams of carbon based products/liter/hour, “gmₚᵣ/l/hr”). Upon triggering of the metabolic switch, m should be reduced to m′ and p should be increased to p′. The majority of the carbon derived from photosynthetically fixed CO₂ that had previously been directed into DCW should be directed into products of interest. Without activation of the metabolic switch, DCW and products of interest would have continued to be produced at rates m and p, respectively, for the duration of the linear phase. In principle, the partitioning of flux between DCW and products of interest, i.e., the m′:p′ ratio, can be quantitatively tuned by controlling the precise concentration of the exogenous compound in the medium that controls the state of the metabolic switch. Any strain carrying out efficient photosynthetic CO₂ fixation has certain minimal growth-independent biomass-maintenance requirements, e.g., photosystem proteins need to be continually recycled due to oxidative damage if photosynthetic electron transport is to be maintained. Thus, solely through modulation of the concentration of the exogenous compound, m′ can be set to the level just required to maintain optimal photosynthesis, thereby maximizing p′.

The operation of a metabolic switch is analyzed with specific reference to a JCC138-derived ethanologen growing in 30 ml JB2.1 medium in a 125 ml unbaffled, foam-plugged flask in a shaking (150 rpm) photoincubator (Multitron II, Infors) set to approximately 100 μmol photons m⁻² s⁻¹ at 37° C. in an air atmosphere enriched with 2% CO₂. In this case, the host strain has been engineered to have the genes, pdc and adhA for synthesizing ethanol, and engineered to be an lplA⁺-lipA lipoic acid auxotroph such that lipoic acid is the exogenous critical compound whose concentration controls the state of the pyruvate dehydrogenase/pyruvate decarboxylase metabolic switch.

The values of m, p, m′, and p′ are derived assuming that the total (DCW+ethanol) energetic productivity of each host cell is the same as the DCW productivity of wild-type JCC138, energetic productivity being invoked because of the different heating values of DCW (measured to be 21.0 kJ/gm_{dcw}) and ethanol (29.0 kJ/g). The total energetic productivity is thus assumed to be a constant at 0.036 gm_{dcw} (wt)/1/hr*21.0 kJ/gm_{dcw} (wt)=756 J/l/hr. This means, for example, that the maximal ethanol productivity is 756/29000=0.0261 g-ethanol/l/hr. The yields are converted to energetic yields, and correspond to the fraction of the 756 J/l/hr that corresponds to either DCW or EtOH. For example, for a post-metabolic-switch phase, if m′=1.8 mg/l/hr and p′=24.8 mg/l/hr, the DCW yield is (0.0018*21000)/756=5%, and the EtOH yield is (0.0248*29000)/756=95%. Yield values are directly derived from productivity values.

To experimentally validate the function of the lipoic-acid-based metabolic switch mechanism, the lplA⁺-lipA lipoic acid auxotrophic JCC138-derived ethanologen is cultured in flasks under culturing conditions specified below, one set of replicate cultures grown in medium containing an excessive amount of lipoic acid (Set I), and the other in medium containing a concentration of lipoic acid designed to become depleted due to cell growth at some time during the linear phase (Set II). For each set of flasks, pdc and adhA are fully induced in the same manner to ensure ethanol synthesis; for example, an engineered Synochococcal host cell with pdc under control of the urea-repressible, nitrate-inducible P(nir07) promoter, this will mean depleting the urea in the urea+nitrate medium. Once the cultures have reached the linear growth phase, samples are taken. For each sample, the OD₇₃₀ is measured spectrophotometrically and converted to gm_{dcq}/l, via the previously determined conversion factor of 0.3 gm_{dcw}/l per 1 OD₇₃₀ unit, and the ethanol titer is measured by gas chromatography coupled with flame ionization detection. Ethanol titers are converted to cumulative ethanol concentrations to account for the stripping of ethanol that occurs during the course of the flask cultivation, via the previously determined ethanol stripping rate constant of 0.006 hr⁻¹. The linear-fit slopes of the gm_{dcw}/l versus time and grams of cumulative ethanol/liter versus time profiles, both before and after the time corresponding to the point of lipoic acid depletion in the Set II flasks, are used to calculate m, p, m′, and p′ for the Set I (_I) and Set II (_II) replicate flask cultures. The metabolic switch is considered functional when, statistically significantly, all of the following are true: (i) m_I=m′_I=m_II; (ii) p_I=p′_I=p_II; (iii) m_II>m′_II; and (iv) p_II<p′_II.

### Example 7

### Evaluation of Productivity Changes by Metabolic Switch in a Photobioreactor Cultured Host Cell

Biosynthetic productivity and photosynthetic efficiency of host cells with and without a metabolic switch are evaluated in a solar converter (a photobioreactor). Specifically, productivity is determined by maintening a constant liquid media-based host cell density and measuring cumulative biosynthesized carbon-product output. To accomplish this, a turbidostat is incorporated into the solar converter culturing device to measure the optical density (0D₇₃₀) of the cultured media. As an OD₇₃₀ becomes higher than a pre-determined set point, cultured cells plus media are released from the solar converter as new “dilution” media without cultured cells is added. Therefore, the growth conditions with respect to OD₇₃₀ are kept constant throughout the life-span of the culture run. Thus, when the flow rate of the dilution media is measured, the OD₇₃₀ of the exit media is known (and the biomass per OD₇₃₀ also is pre-determined), the operator can calculate the culture growth rate as biomass produced per unit of time. Constant OD₇₃₀ is maintained by using a Metler Toledo Turbidity probe (model #8300) to monitor OD₇₃₀, with which data output is processed and interfaced with a peristaltic pump. When the culture grows above a pre-determined OD₇₃₀ set-point, the pump is activated and adds filter sterilized JB 2.1 media. An equal amount of excess culture drains out of a separate port in the reactor, allowing both the volume and OD₇₃₀ in the reactor to be maintained at a constant value. The optical density is confirmed by taking manual samples and measuring them on a spectrophotometer (wavelength 730 nm).

Pump activity is recorded over time by measuring weight changes of the media bottle, which rests on a Sartorius Signum scale. A Winwedge program saves data from the scale every five minutes to data file. As applied herein, the weight is directly converted to volume because the media density is approximately 1.0 g/mL.

Total light energy input required for biomass and carbon-based product biosynthesis for a particular culture run is calculated from known parameters of the photobioreactor, including total photobioreactor area exposed to a light source and total photon flux provided by the light source for the duration of the culture run. Once these parameters are evaluated, photosynthetic efficiency can be determined before and after a metabolic switch is incorporated into the host cell.

Productivity is calculated using equations 7-11:

Biomass Areal Productivity=(OD_{730b}/3.3)(fᵣ)(α) Eqn. 7

where biomass areal productivity is in units of grams/meter²/hour, “3.3” is an empircle value of host cell mass per unit OD₇₃₀ and is in grams/liter, fᵣ is peristaltic pump flow rate in liters/hour, and cc is inverse of the reactor area.

Ethanol Productivity in Liquid Culture=(Et_{c})(fᵣ)(α) Eqn. 8

where ethanol productivity in liquid culture is in units of grams/meter²/hour and Et_{c} is ethanol concentration in units of grams/liter as measured evaluated by gas chromatography analysis.

Ethanol Stripped from Culture=(Et_{c})(ω)(V)(α) Eqn. 9

where ω is the stripping rate in units of hour⁻¹ and is specific to the photobioreactor, and V is total functioning volume of the photobioreactor in units of liters.

Total Areal Ethanol Productivity=Eqn. 8+Eqn. 9 Eqn. 10

where total areal ethanol productivity is in units of grams/meter²/hour.

Total Ethanol Yield=Eqn. 10/(Eqn. 7+Eqn. 10). Eqn. 11

Photosynthetic efficiency is calculated in equations 12-16:

Eₚₕ=h(c/λ) Eqn. 12

where Eₚₕ is the energy of one photon in units of Joule, h is Planck's constant equal to 6.626 e⁻³⁴ Joule seconds, λ is the average photosynthetically active radiation (PAR) wavelength of the light source in units of nanometers, and c is the speed of light in units of nanometers/second (3e¹⁷ nm/s).

Hourly Light Energy Input=(Eqn. 7)(I)(E/10e⁶ μE)N_{A}⁻¹(3600 sec/hr) Eqn. 13

where hourly light energy input is in units of Joules/meter²/second, I is intensity in units of μEinsteins/meter²/second, N_{A} is Avogadro's constant (6.022e²³ photons/Einstein) and E is an Einstein (equal to 1 mole of photons).

Total biomass energy(E_{b})=(Eqn. 7)(kg/1000 g)(δ_{biomass}) Eqn. 14

where δ_{biomass} is the energy content of biomass, empirically determined and specific to host cell type, in units of mega-Joules/kilogram.

Total ethanol energy(E_{EtOH})=(Eqn. 5)(kg/1000 g)(δ8_{EtOH}) Eqn. 15

where δ_{EtOH} is the total energy content of ethanol, empirically determined to be 29.7 mega-Joules/kilogram.

θ=(Eqn. 8)/(Eqn. 9+Eqn. 10) Eqn. 16

where θ=photosynthetic efficiency.

### Informal Sequence Listing

SEQ ID NO. 1:
5′GCTTGTAGCAATTGCTACTAAAAACTGCGATCGCTGCTGAAATGAGCT
GGAATTTTGTCCCTCTCAGCTCAAAAAGTATCAATGATTACTTAATGTTT
GTTCTGCGCAAACTTCTTGCAGAACATGCATGATTTACAAAAAGTTGTAG
TTTCTGTTACCAATTGCGAATCGAGAACTGCCTAATCTGCCGAGTATGCG
ATCCTTTAGCAGGAGGAAAACCATATG 3′

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. A method for biosynthesis of carbon-based products of interest in an engineered host cell, comprising:
a. culturing an engineered host cell, wherein said engineered host cell is auxotrophic for at least one exogenous compound, and wherein said engineered host cell comprises at least one control element, at least one heterologous metabolic pathway, at least one second metabolic pathway, and a shared metabolic junction, wherein said exogenous compound controls the activity of a control element and said control element controls carbon flux through said metabolic junction to said heterologous metabolic pathway or to said second metabolic pathway, and wherein said culturing is in the presence of said exogenous compound; and
b. depleting said exogenous compound from the culture.

105. The method of claim 104, wherein said exogenous compound is lipoic acid.

106. The method of claim 104, wherein said depletion of said exogenous compound from said culture increases said carbon flux through said metabolic junction to said heterologous metabolic pathway.

107. The method of claim 104, wherein said at least one second metabolic pathway is an engineered metabolic pathway.

108. The method of claim 104, wherein said engineered host cell attenuates acetyl-CoA production upon depletion of said exogenous compound.

109. The method of claim 104, wherein said engineered host cell attenuates acetyl-CoA production and initiates ethanol production concomitant with depletion of said exogenous compound.

110. The method of claim 104, wherein said heterologous metabolic pathway of said engineered host cell comprises a heterologous alcohol dehydrogenase (“Adh”) and a heterologous pyruvate decarboxylase (“Pdc”).

111. The method of claim 104, wherein said engineered host cell further comprises attenuated pyruvate formate lyase, lactate dehydrogenase, pyruvate:ferredoxin oxidoreductase, or combinations thereof.

112. The method of claim 104, wherein said engineered host cell further comprises a heterologous lipoylation gene product.

113. The method of claim 112, wherein said heterologous lipoylation gene product is selected from the group consisting of: Escherichia coli LplA; lipoyl (octanoyl) transferase (EC 2.3.1.181); and lipoyl synthase (EC 2.8.1.8).

114. The method of claim 112, wherein said engineered host cell further comprises attenuated acyl-ACP synthetase (EC 6.2.1.20), LipA1, LipA2, LipB, or any combination thereof.

115. The method of claim 104, wherein said engineered host cell further comprises a heterologous lipoamidase (“Lpa”).

116. The method of claim 104, wherein said host cell further comprises expression control of a citrase synthase gene.

117. The method of claim 116, wherein said citrase synthase gene is encoded by SEQ ID NO: 1 and is under the control of a heterologous nitrite reductase promoter.

118. The method of claim 104, wherein said carbon based product of interest is ethanol.

119. The method of claim 118, wherein said engineered host cell cultured in the absence of said exogenous compound produces at least the same amount of ethanol as said engineered host cell cultured in the presence of said exogenous compound.

120. The method of claim 118, wherein said engineered host cell comprises one or more recombinant genes affecting carbon flux through said heterologous pathway, and wherein said engineered host cell produces ethanol at an equal or greater maximum rate than an identical background host cell cultured under identical conditions, but lacking said one or more recombinant genes.

121. The method of claim 118, wherein said engineered host cell further comprises an ethanol production rate during a transition mid-point between a linear growth phase and a stationary growth phase which is at least as high as the ethanol production rate of said engineered host cell during said linear growth phase.

122. The method of claim 104, wherein said engineered host cell is a cyanobacterium.

123. A host cell comprising a control element and a heterologous metabolic pathway, wherein said host cell is auxotrophic for an exogenous compound, wherein said exogenous compound controls activity of said control element, and wherein said control element controls carbon flux through a metabolic junction shared by said heterologous metabolic pathway and at least one second metabolic pathway.


SEQUENCE LISTING (4 sequences)

SEQ ID NO: 1 - DNA, 225 bp, Artificial Sequence
  Description of Artificial Sequence Synthetic polynucleotide
gcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatg

SEQ ID NO: 2 - protein, 725 aa, Enterococcus faecalis
MLAQESILETTVQTETESVTTETSQTVANLESETTSQTVMQEKESSSAIAESSSRNVVAVTTETTNEIQNSGTDGKAVSAESVFSEADYKQATALELATLVREKKVTSEELVKIALAITKRENPTLNAVITLREEAALTEAKALQDTGQPFLGVPLLLKGLGQSLKGESNTNGFGFLRDQVAGGTSTFVKALQNAGFIIIGQTNYPELGWKNISDSKLYGVSVNPWNPNHYSGGSSGGAGASVAAAFVPIASGSDAGGSIRIPASWTGTVGLKPSRGVIIGNSNSAKGQTVHFGLSRTVADTNALFETLLTKKDLPAGHLSQAQPIAYTTESPAGTPVSAEAKEAVAEAVAFLKDQGYTLVEVKHPVDGERLMKNYYTVAAGSAGIADFMARQKLKRPLERNDVELLTWALFQTGKNITSEETTAAWTDIALQAQAMDEFYQQYPILLTPTTAATAPSIDNPLLKPEHAAQMEKIDQLSPAEQKQLIYDQWLTAFTYTPFTQQANLFGHPALSVPTYVSKEGLPLGIQFNSALNEDRTLLQLGALFENNHKINQPHVEEPDKDKEPDASGEPEKDKDPNASGEPDKDKEPDASGEPDKDKEPDASGEPDKDKEPDASGKPDKDKETKTSEGPIEGKDQNQNPDKAGKTTSGSSLDNSLNSSANQGTKSTESTHAFSNKSMIGKQEQLPKKVLPKAGAEVPSTFWIVLGGAFLVTSGTIYIRKTRK

SEQ ID NO: 3 - protein, 490 aa, Enterococcus faecalis
  Sequence is truncated at any one of amino acids at positions 450-490
MLAQESILETTVQTETESVTTETSQTVANLESETTSQTVMQEKESSSAIAESSSRNVVAVTTETTNEIQNSGTDGKAVSAESVFSEADYKQATALELATLVREKKVTSEELVKIALAITKRENPTLNAVITLREEAALTEAKALQDTGQPFLGVPLLLKGLGQSLKGESNTNGFGFLRDQVAGGTSTFVKALQNAGFIIIGQTNYPELGWKNISDSKLYGVSVNPWNPNHYSGGSSGGAGASVAAAFVPIASGSDAGGSIRIPASWTGTVGLKPSRGVIIGNSNSAKGQTVHFGLSRTVADTNALFETLLTKKDLPAGHLSQAQPIAYTTESPAGTPVSAEAKEAVAEAVAFLKDQGYTLVEVKHPVDGERLMKNYYTVAAGSAGIADFMARQKLKRPLERNDVELLTWALFQTGKNITSEETTAAWTDIALQAQAMDEFYQQYPILLTPTTAATAPSIDNPLLKPEHAAQMEKIDQLSPAEQKQLIYDQ

SEQ ID NO: 4 - protein, 471 aa, Enterococcus faecalis
MLAQESILETTVQTETESVTTETSQTVANLESETTSQTVMQEKESSSAIAESSSRNVVAVTTETTNEIQNSGTDGKAVSAESVFSEADYKQATALELATLVREKKVTSEELVKIALAITKRENPTLNAVITLREEAALTEAKALQDTGQPFLGVPLLLKGLGQSLKGESNTNGFGFLRDQVAGGTSTFVKALQNAGFIIIGQTNYPELGWKNISDSKLYGVSVNPWNPNHYSGGSSGGAGASVAAAFVPIASGSDAGGSIRIPASWTGTVGLKPSRGVIIGNSNSAKGQTVHFGLSRTVADTNALFETLLTKKDLPAGHLSQAQPIAYTTESPAGTPVSAEAKEAVAEAVAFLKDQGYTLVEVKHPVDGERLMKNYYTVAAGSAGIADFMARQKLKRPLERNDVELLTWALFQTGKNITSEETTAAWTDIALQAQAMDEFYQQYPILLTPTTAATAPSIDNPLLKPEHAAQ